(19) United States
(12) Patent Application Publication
(10) Pub. No.: US 2024/0344112 A1
(43) Pub. Date: Oct. 17, 2024

(54) ITERATIVE OLIGONUCLEOTIDE BARCODE EXPANSION FOR LABELING AND LOCALIZING MANY BIOMOLECULES

(71) Applicant: GOOGLE LLC, Mountain View, CA (US)

(72) Inventors: Ali BASHIR, San Mateo, CA (US); Marc BERNDL, Mountain View, CA (US); Annalisa PAWLOSKY, Mountain View, CA (US); Jun KIM, Mountain View, CA (US); Sara AHADI, Mountain View, CA (US); Alexander TRAN, Mountain View, CA (US)

(21) Appl. No.: 18/580,283

(22) PCT Filed: Jul. 20, 2022
(86) PCT No.: PCT/US2022/037673

§ 371 Date: Jan. 18, 2024

Related U.S. Application Data
(60) Provisional application No. 63/224,295, filed on Jul. 21, 2021.

(51) Int. Cl. C12Q 1/6806; C12Q 1/6869

(52) U.S. Cl. CPC C12Q 1/6806; C12Q 1/6869

(57) ABSTRACT

Contemporary gene sequencing techniques, including “Next. Generation Sequencing” techniques, can include sequencing a plurality of fragments of a target polynucleotide. However, the limitations of existing sequencing techniques means that it can be difficult and/or expensive to align the generated read fragments. Methods provided herein include inserting dual polynucleotide ‘bar-codes’ into a target poly nucleotide that remain mechanically connected via a Tinker.’ Tire barcodes can then be ‘grown’ via. a. pool-split-pool process such that polynucleotide fragments that are linked by linkers exhibit the same complete barcode sequence that is different, from the complete barcode sequence exhibited by non-linked polynucleotide fragments. The joined fragments can then be separated and sequenced. Each read sequence thus begins with a regionally-specific barcode that can be used to associate fragments from the region together, allowing for increased accuracy and reduced computational cost in aligning the read fragments and/or performing other sequencing processes on the read fragments.

### CROSS-REFERENCE TO RELATED APPLICATION

This application claims priority to U.S. Provisional Application No. 63/224,295, filed Jul. 21, 2021, which is hereby incorporated by reference in its entirety.

### BACKGROUND

Early DNA sequencing techniques, such as chain-termination methods, provided reliable solutions for reading individual DNA fragments See Sanger, F. et. al. (1977) DNA sequencing with chain-terminating inhibitors. Proc. Natl. Acad. Sci. U.S.A. 74, 5463-5467. While these first-generation technologies are effective for sequencing target genes, applying them to sequencing entire chromosomes or genomes is costly and expensive. For example, the first sequencing of a human genome—which was accomplished using the Sanger method—cost hundreds of millions of dollars and took over a decade to complete. This high cost was largely due to the sequential nature of first-generation sequencing methods; each fragment had to be individually read and manually assembled to construct a full genome.

Next generation sequencing (NGS) technologies have significantly reduced the cost of DNA sequencing by parallelizing DNA fragment reading. Some NGS methods are capable of performing millions of sequence reads concurrently, generating data for millions of base pairs in a matter of hours. See Hall, N. (2007) Advanced sequencing technologies and their wider impact in microbiology. The Journal of Experimental Biology, 209, 1518-1525. Many NGS technologies have been proposed, and employ various chemical processes, use varying read lengths, and have demonstrated various ranges of accuracy. See Metzker, M. (2010) Sequencing technologies—the next generation. Nature Reviews, Genetics, Volume 11, 31-46; see also Shendure, J. et. al. (2008) Next-generation DNA sequencing. Nature Reviews, Biotechnology, Volume 26, Number 10, 1135-1145.

NGS methods generally involve separating a DNA sample into fragments and reading the nucleotide sequence of those fragments in parallel. The resulting data generated from this process includes read data for each of those fragments, which contains a continuous sequence of nucleotide base pairs (G, A, T, C). However, while the arrangement of base pairs within a given fragment read is known, the arrangement of the fragment reads with respect to each other is not. Thus, to determine the sequence of a larger DNA strand (such as a gene or chromosome), read data from multiple fragments must be aligned. This alignment is relative to other read fragments, and may include overlapping fragments. depending upon the particular NGS method used. Some NGS methods use computational techniques and software tools to carry out read data alignment.

Accurate sequence read alignment is the first step in identifying genetic variations in a sample genome. The diverse nature of genetic variation can cause alignment algorithms and techniques to align sequence reads to incorrect locations within the genome. Furthermore, the read process used to generate sequence reads may be complex and susceptible to errors. Thus, many sequence read alignment techniques can misalign a sequence read within a genome, which can lead to incorrect detection of variants in subsequent analyses.

Once the read data has been aligned, that aligned data may be analyzed to determine the nucleotide sequence for a gene locus, gene. or an entire chromosome. However, differences in nucleotide values among overlapping read fragments may be indicative of a variant, such as a single-nucleotide polymorphism (SNP) or an insertion or deletion (INDELs), among other possible variants. For example, if read fragments that overlap at a particular locus differ, those differences might be indicative of a heterozygous SNP. As another example, if overlapping read fragments are the same at a single nucleotide, but differ from a reference genome, that gene locus or gene may be a homozygous SNP with respect to that reference genome. Accurate determination of such variants is an important aspect of genome sequencing, since those variants could represent mutations, genes that cause particular diseases, and/or otherwise serve to genotype a particular DNA sample.

The demand for high efficiency and low-cost DNA sequencing has increased in recent years. Although NGS technologies have dramatically improved upon first-generation technologies, the highly-parallelized nature of NGS techniques has presented challenges not encountered in earlier sequencing technologies. Errors in the read process can adversely impact the alignment of the resulting read data, and can subsequently lead to inaccurate sequence determinations. Furthermore, read errors can lead to erroneous detection of variants.

A more comprehensive and accurate understanding of both the human genome as a whole and the genomes of individuals can improve medical diagnoses and treatment. NGS technologies have reduced the time and cost of sequencing an individual's genome, which provides the potential for significant improvements to medicine and genetics in ways that were previously not feasible. Understanding genetic variation among humans provides a framework for understanding genetic disorders and Mendelian diseases. However, discovering these genetic variations depends upon reliable read data and accurate read sequence alignment.

### SUMMARY

In a first aspect, a method is provided that includes: (i) adding a probe to a sample that contains a target polynucleotide, wherein the probe includes (a) a first payload polynucleotide, (b) a second payload polynucleotide, (c) a linker that links the first payload polynucleotide to the second payload polynucleotide, and (d) an insertion vector, and wherein the insertion vector inserts the first payload polynucleotide and second payload polynucleotide into the target polynucleotide, thereby fragmenting the target polynucleotide into a portion that terminates with the first payload polynucleotide and another portion that terminates with the second payload polynucleotide and that is linked, via the linker, to the portion that terminates with the first payload polynucleotide; (ii) fragmenting the target polynucleotide; (iii) subsequent to fragmenting the target polynucleotide, splitting the sample into two or more split samples; (iv) adding a first barcoding agent to a first split sample of the two or more split samples, wherein the first barcoding agent extends instances of the first payload polynucleotide and the second payload polynucleotide in the first split sample to include a first polynucleotide barcode; (v) adding a second barcoding agent to a second split sample of the two or more split samples, wherein the second barcoding agent extends instances of the first payload polynucleotide and the second payload polynucleotide in the second split sample to include a second polynucleotide barcode, and wherein the second polynucleotide barcode differs from the first polynucleotide barcode; (vi) pooling the two or more split samples into a pooled sample; and (vii) subsequent to pooling the two or more split samples, severing instances of the linker, thereby decoupling instances of the first polynucleotide barcode from associated instances of the second polynucleotide barcode.

The method could additionally include: splitting the pooled sample into two or more additional split samples; adding a third barcoding agent to a third split sample of the two or more additional split samples, wherein the third barcoding agent extends instances of the first payload polynucleotide and the second payload polynucleotide in the third split sample to include a third polynucleotide barcode; and adding a fourth barcoding agent to a fourth split sample of the two or more additional split samples, wherein the fourth barcoding agent extends instances of the first payload polynucleotide and the second payload polynucleotide in the fourth split sample to include a fourth polynucleotide barcode, and wherein the fourth polynucleotide barcode differs from the third polynucleotide barcode. In some examples, the first payload polynucleotide and the second payload polynucleotide of the probe each end in a first recognition sequence; the first barcoding agent specifically targets the first recognition sequence to extend instances of the first payload polynucleotide and the second payload polynucleotide in the first split sample to include the first polynucleotide barcode and to end in a second recognition sequence; the second barcoding agent specifically targets the first recognition sequence to extend instances of the first payload polynucleotide and the second payload polynucleotide in the second split sample to include the second polynucleotide barcode and to end in the second recognition sequence; the third barcoding agent specifically targets the second recognition sequence to extend instances of the first payload polynucleotide and the second payload polynucleotide in the third split sample to include the third polynucleotide barcode; and the fourth barcoding agent specifically targets the second recognition sequence to extend instances of the first payload polynucleotide and the second payload polynucleotide in the fourth split sample to include the fourth polynucleotide barcode.

The method could additionally include, prior to splitting the pooled sample into two or more additional split samples, fragmenting the target polynucleotide in the pooled sample.

In some examples, (i) the first payload polynucleotide and the second payload polynucleotide of the probe each end in a first recognition sequence, (ii) the first barcoding agent specifically targets the first recognition sequence to extend instances of the first payload polynucleotide and the second payload polynucleotide in the first split sample to include the first polynucleotide barcode, and (iii) the second barcoding agent specifically targets the first recognition sequence to extend instances of the first payload polynucleotide and the second payload polynucleotide in the second split sample to include the second polynucleotide barcode. For example, the probe can additionally include a third payload polynucleotide that is associated with the first payload polynucleotide as double-stranded DNA and a fourth payload polynucleotide that is associated with the second payload polynucleotide as double-stranded DNA; the insertion vector ligates the first payload polynucleotide to a 3′ end of a first strand of the target polynucleotide and ligates the third payload polynucleotide to a 5′ end of a second strand of the target polynucleotide; and a portion of a 3′ end of the first payload polynucleotide that includes the first recognition sequence extends beyond a 5′ end of the third payload polynucleotide.

In some examples. the linker comprises polyethylene glycol with a length between 40 monomer subunits and 125 monomer subunits.

In some examples, the first payload polynucleotide includes a modified nucleotide via which the first payload polynucleotide is linked to the linker, and severing instances of the linker comprises chemically reacting the modified nucleotide to decouple the first payload polynucleotide from the linker.

In some examples, the first barcoding agent includes T7 ligase and extends instances of the first payload polynucleotide and the second payload polynucleotide in the first split sample by ligating the first polynucleotide barcode to exposed ends of the first payload polynucleotide and the second payload polynucleotide.

In some examples, the insertion vector of an individual instance of the probe comprises a first Tn5 transposase coupled to the first payload polynucleotide and a second Tn5 transposase coupled to the second payload polynucleotide.

The method could additionally include, subsequent to severing instances of the linker, sequencing a plurality of segments of the target polynucleotide that include at least one of an instance of the first payload polynucleotide or an instance of the second payload polynucleotide to obtain reads of the fragments of the target polynucleotide; and determining a sequence for the target polynucleotide based on the reads of the fragments of the target polynucleotide, wherein determining the sequence for the target polynucleotide comprises: identifying a regional barcode for each of the read fragments of the target polynucleotide, wherein the regional barcode for a read fragment obtained from a fragment of the target polynucleotide that was present in the first split sample includes the first polynucleotide barcode, and wherein the regional barcode for a read fragment obtained from a fragment of the target polynucleotide that was present in the second split sample includes the second polynucleotide barcode; and associating sets of the read fragments together based on correspondences between their respective identified regional barcodes.

In some examples, the target polynucleotide comprises DNA.

In some examples, the target polynucleotide comprises RNA; the target polynucleotide is a first isoform of an RNA sequence; and the sample contains a second isoform of the RNA sequence, and wherein the first isoform differs from the second isoform.

In another aspect, a probe is provided that includes: (i) a first payload polynucleotide; (ii) a second payload polynucleotide; (iii) a linker that links the first payload polynucleotide to the second payload polynucleotide; and (iv) an insertion vector, wherein the insertion vector inserts the first payload polynucleotide and second payload polynucleotide into the target polynucleotide, thereby fragmenting the target polynucleotide into a portion that terminates with the first payload polynucleotide and another portion that terminates with the second payload polynucleotide and that is linked. via the linker, to the portion that terminates with the first payload polynucleotide.

In some examples, the insertion vector comprises a first Tn5 transposase coupled to the first payload polynucleotide and a second Tn5 transposase coupled to the second payload polynucleotide.

In some examples, the linker comprises polyethylene glycol with a length between 40 monomer subunits and 125 monomer subunits

In some examples, the first payload polynucleotide includes a modified nucleotide via which the first payload polynucleotide is linked to the linker.

In some examples, the probe additionally comprises a third payload polynucleotide that is associated with the first payload polynucleotide as double-stranded DNA and a fourth payload polynucleotide that is associated with the second payload polynucleotide as double-stranded DNA; the insertion vector ligates the first payload polynucleotide to a 3′ end of a first strand of the target polynucleotide and ligates the third payload polynucleotide to a 5′ end of a second strand of the target polynucleotide, and a portion of a 3′ end of the first payload polynucleotide that includes a first recognition sequence extends beyond a 5′ end of the third payload polynucleotide.

In yet another aspect, a method is provided that includes: (i) adding a plurality of instances of a probe to a target polypeptide in a sample, wherein each instance of the probe is coupled to the target polypeptide at a respective different amino acid of the target polypeptide, and wherein the probe comprises a payload polynucleotide; (ii) splitting the sample into two or more split samples; (iii) adding a first barcoding agent to a first split sample of the two or more split samples, wherein the first barcoding agent extends instances of the payload polynucleotide in the first split sample to include a first polynucleotide barcode; (iv) adding a second barcoding agent to a second split sample of the two or more split samples, wherein the second barcoding agent extends instances of the payload polynucleotide in the second split sample to include a second polynucleotide barcode, and wherein the second polynucleotide barcode differs from the first polynucleotide barcode; (v) pooling the two or more split samples into a pooled sample: (vi) subsequent to pooling the two or more split samples, fragmenting the target polypeptide, thereby generating a set of fragments of the target polypeptide with each fragment of the target polypeptide coupled to a respective instance of the payload polynucleotide that has been extended to include at least one polynucleotide barcode; and (vii) obtaining, for each fragment of the target polypeptide, a sequence read for a fragment of the target polypeptide and a sequence read for an extended payload polynucleotide coupled thereto.

The method could additionally include: splitting the pooled sample into two or more additional split samples; adding a third barcoding agent to a third split sample of the two or more additional split samples, wherein the third barcoding agent extends instances of the payload polynucleotide in the third split sample to include a third polynucleotide barcode; and adding a fourth barcoding agent to a fourth split sample of the two or more additional split samples, wherein the fourth barcoding agent extends instances of the payload polynucleotide in the fourth split sample to include a fourth polynucleotide barcode, and wherein the fourth polynucleotide barcode differs from the third polynucleotide barcode.

In some examples, the payload polynucleotide ends in a first recognition sequence; the first barcoding agent specifically targets the first recognition sequence to extend instances of the payload polynucleotide in the first split sample to include the first polynucleotide barcode and to end in a second recognition sequence; the second barcoding agent specifically targets the first recognition sequence to extend instances of the payload polynucleotide in the second split sample to include the second polynucleotide barcode and to end in the second recognition sequence; the third barcoding agent specifically targets the second recognition sequence to extend instances of the payload polynucleotide in the third split sample to include the third polynucleotide barcode; and the fourth barcoding agent specifically targets the second recognition sequence to extend instances of the payload polynucleotide in the fourth split sample to include the fourth polynucleotide barcode.

In some examples. the payload polynucleotide ends in a first recognition sequence; the first barcoding agent specifically targets the first recognition sequence to extend instances of the payload polynucleotide in the first split sample to include the first polynucleotide barcode; and the second barcoding agent specifically targets the first recognition sequence to extend instances of the payload polynucleotide in the second split sample to include the second polynucleotide barcode.

In some examples, the payload polynucleotide is associated with a complementary polynucleotide as double-stranded DNA; and a portion of a 3′ end of the first payload polynucleotide that includes the first recognition sequence extends beyond a 5′ end of the complementary polynucleotide.

In some examples, the payload polynucleotide comprises a segment of single-stranded DNA that is coupled to the target polypeptide via a 3′ end; and the first barcoding agent extends instances of the payload polynucleotide in the first split sample to include a first polynucleotide barcode by ligating a 3′ end of the first polynucleotide barcode to a 5′ end of the target polypeptide.

In some examples, the payload polynucleotide comprises a restriction sequence; and the method further comprises, subsequent to fragmenting the target polypeptide, fragmenting the extended payload polynucleotide at the restriction sequence, thereby decoupling a portion of the extended payload polynucleotide that has been extended to include at least one polynucleotide barcode from an associated fragment of the target polypeptide.

The method could additionally include: extending instances of the payload polynucleotide to include a linker; and subsequent to fragmenting the target polypeptide and prior to fragmenting the extended payload polynucleotide at the restriction sequence, (i) coupling a fragment of the target polypeptide to a support via an amino acid of the fragment, and (ii) coupling an extended payload polynucleotide that is coupled to the fragment of the target polypeptide to the support via the linker.

In some examples, the first barcoding agent includes T7 ligase and extends instances of the payload polynucleotide in the first split sample by ligating the first polynucleotide barcode to an exposed end of the payload polynucleotide.

The method could additionally include: subsequent to obtaining, for each fragment of the target polypeptide, a sequence read for the fragment of the target polypeptide and a sequence read for the extended payload polynucleotide coupled thereto, determining a sequence for the target polypeptide based on the sequence reads of the fragments of the target polypeptide, wherein determining the sequence for the target polypeptide comprises: identifying a regional barcode for each of the sequence reads of the extended payload polynucleotides, wherein the regional barcode for a sequence read obtained from an extended payload polynucleotide that was present in the first split sample includes the first polynucleotide barcode, and wherein the regional barcode for a sequence read obtained from an extended payload polynucleotide that was present in the second split sample includes the second polynucleotide barcode. and associating sets of sequence reads for the fragments of the target polypeptide together based on correspondences between regional barcodes identified in the extended payload polynucleotides associated therewith.

In some examples, fragmenting the target polypeptide comprises fragmenting the target polypeptide such that each instance of the payload polynucleotide that has been extended to include at least one polynucleotide barcode is coupled to a respective fragment of the target polypeptide via a first terminal amino acid of the fragment of the target polypeptide.

In some examples, obtaining a sequence read for a particular fragment of the target polypeptide comprises: coupling the particular fragment to a support; adding, to an extended payload polynucleotide that is associated with the particular fragment, a polynucleotide sequence indicative of an identity of at least one amino acid at an end of the particular fragment opposite the first terminal amino acid of the particular fragment; and, subsequent to adding the polynucleotide sequence indicative of the identity of the at least one amino acid at the end of the particular fragment opposite the first terminal amino acid, removing from the particular fragment at least one amino acid from the end of the particular fragment opposite the first terminal amino acid.

In some examples, adding the polynucleotide sequence indicative of an identity of at least one amino acid at an end of the particular fragment opposite the first terminal amino acid of the particular fragment comprises: adding, to a sample that includes the support, an aptamer that selectively binds to polypeptides that terminate in the at least one amino acid that comprise the end of the particular fragment opposite the first terminal amino acid of the particular fragment, wherein the aptamer also comprises the sequence indicative of the identity of the at least one amino acid at the end of the particular fragment opposite the first terminal amino acid of the particular fragment; and fragmenting, from the remainder of the aptamer, the sequence indicative of the identity of the at least one amino acid at the end of the particular fragment opposite the first terminal amino acid of the particular fragment.

In some examples, the payload polynucleotide comprises a restriction sequence, and the method further comprises: coupling an extended payload polynucleotide that is coupled to the particular fragment to the support; and fragmenting the extended payload polynucleotide that is coupled to the particular fragment at the restriction sequence, thereby decoupling a portion of the extended payload polynucleotide that has been extended to include at least one polynucleotide barcode from the particular fragment.

In some examples, the first terminal amino acid of the particular fragment is located at a C-terminus of the particular fragment, and wherein removing from the particular fragment at least one amino acid from the end of the particular fragment opposite the first terminal amino acid comprises performing an Edman degradation.

In yet another aspect, as a non-transitory computer readable medium is provided having stored therein instructions executable by a computing device to cause the computing device to perform any of the above methods.

The foregoing summary is illustrative only and is not intended to be in any way limiting. In addition to the illustrative aspects, embodiments, and features described above, further aspects, embodiments, and features will become apparent by reference to the figures and the following detailed description and the accompanying drawings.

### BRIEF DESCRIPTION OF THE FIGURES

FIG. 1 illustrates aspects of an example method for barcoding polynucleotides.

FIG. 2A illustrates aspects of an example method for barcoding polynucleotides.

FIG. 2B illustrates aspects of an example method for barcoding polynucleotides.

FIG. 2C illustrates aspects of an example method for barcoding polynucleotides.

FIG. 3A depicts experimental results.

FIG. 3B depicts experimental results.

FIG. 4 illustrates aspects of an example method for barcoding polypeptides.

FIG. 5A illustrates aspects of an example method for sequencing polypeptides.

FIG. 5B illustrates aspects of an example method for sequencing polypeptides.

FIG. 6 illustrates a flowchart of an example method.

FIG. 7 illustrates a flowchart of an example method.

### DETAILED DESCRIPTION

The following detailed description describes various features and functions of the disclosed systems and methods with reference to the accompanying figures. The illustrative system and method embodiments described herein are not meant to be limiting. It may be readily understood that certain aspects of the disclosed systems and methods can be arranged and combined in a wide variety of different configurations, all of which are contemplated herein.

### I. Overview

Next Generation Sequencing methods or other modern sequencing techniques have enabled many applications by reducing the cost to sequence samples of DNA or other polynucleotides (e.g., RNA, synthetic polynucleotides). These techniques generally include determining the sequence of hundreds, thousands, or more fragments of a target sample and then performing alignment and/or other computational processes on the fragment sequences in order to determine the sequence of the target sample. This computational process is difficult and can be computationally intensive. Additionally, the presence of repeating sequences at a single location within the target, duplicated sequences at different locations within the target. imperfections in the fragment sequencing process, and other factors can mean that, in some circumstances, the available fragment sequences do not permit perfect and unambiguous reconstruction of the sequence of the target.

The methods described herein improve the process of sequencing a target polynucleotide in a sample by fragmenting the target while keeping fragments that are nearby tethered together. Each assembly of tethered-together fragments can then be ‘grown,’ via serial ligation of short barcode sequences, to terminate in a polynucleotide barcode sequence that is unique to the assembly and shared by each of the fragments in the assembly. A sample containing multiple such assemblies of tethered-together fragments can be subjected to repeated cycles of splitting into separate samples, ligating a different short barcode sequence to fragments in each of the different samples, and pooling the separate samples back together.

Such a repeated split-pool process quickly and cost-effectively grows a unique region-specific barcode (each ‘region’ being the region of the target spanned by the fragments tethered together as part of each assembly) on each of the fragments in the sample. The fragments in each assembly can then be un-tethered (e.g., by using click chemistry to sever the polyethylene glycol chains or other linking agent(s)) and sequenced. The sequence for each fragment will begin with a region-specific fragment for the fragment which can be used to facilitate alignment of the fragments into a reconstructed sequence for the target sample.

This linked-fragment process includes inserting paired polynucleotide ‘end caps,’ that are linked to each other via polyethylene glycol or some other linking agent, into a target polynucleotide a number of times such that the target polynucleotide is fragmented into a number of fragments that terminate in the ‘end caps’ and that are thus tethered to neighboring fragments via the linking agent that links together the ‘end caps.’ These ‘end caps’ can be composed of single-stranded DNA (“ssDNA”), double-stranded DNA (“dsDNA”), RNA, or some other type of polynucleotide that is compatible with being inserted into and/or ligated onto the end of fragments of the target polynucleotide (or that can be converted into such a polynucleotide, e.g., by translating a target RNA into cDNA). The target polynucleotide can then be further fragmented (without tethering/insertion of ‘end caps’) in order to facilitate labeling of different ‘regions’ of the target (which correspond to respective assemblies of tethered-together fragments of the target) via the split-pool process. Additional fragmentation could occur after one or more cycles of the split-pool process, e.g., to allow for ‘sub-regional’ barcoding.

Similar methods of regional barcoding via repeated split-pool barcode growth could also be applied to improve sequencing of proteins or other polypeptides. A base polynucleotide (e.g., a length of double-stranded DNA) could then be attached to a target polypeptide a number of times at a number of locations along the length of the polypeptide (e.g., to every instance of a specified amine within the polypeptide). Each of the attached polynucleotides could then be grown, via a repeated split-pool process, such that each of the polynucleotides attached to a single polypeptide include the same polypeptide-specified barcode sequence. The polypeptide could then be fragmented such that each fragment is attached to a respective instance of the barcoded polynucleotides. The fragments, along with their associated barcode polynucleotides, could then be sequenced and the pairs of sequences (polypeptide fragment sequence and associated barcode polynucleotide sequence) used to reconstruct the complete sequence of the polypeptide (e.g., by associating all of the polypeptide fragment sequences together if they correspond to polynucleotide sequences bearing the same barcode).

Such a polypeptide barcoding and sequencing process could facilitate cheaper, simpler, and/or higher-accuracy sequencing of polypeptides. This could include improving the sequencing of longer polypeptides via length-limited polypeptide sequencing techniques (e.g., Edman degradation). These improvements may be related to the ability to correspond shorter polypeptide fragment sequences together by using correspondences in their associated polynucleotide barcode sequences. In some examples, the polynucleotide barcode could be extended as part of the polypeptide fragment sequencing process to represent the sequence of the polypeptide fragment. Thus, sequencing of the polypeptide fragment, as well as determining the barcode sequence corresponding thereto, can be accomplished by sequencing the extended polynucleotide

### II. Introduction

Next generation sequencing (NGS) has dramatically reduced the time and cost required to sequence an entire genome. Previous techniques involved sequentially reading out DNA fragments and having trained biochemists arrange the read data to determine the sequence of entire chromosomes. NGS technologies parallelize the sequencing process, allowing millions of DNA fragments to be read simultaneously. Automated computational analyses then attempt to align the read data to determine the nucleotide sequence of a gene locus, gene, chromosome, or entire genome.

The increasing prevalence of NGS technologies has generated a substantial amount of genome data. Analysis of this genome data—both for an individual sample and for multiple samples—can provide meaningful insights about the genetics of a sample (e.g., an individual human patient) or species. Variations between genomes may correspond to different traits or diseases within a species. Variations may take the form of single nucleotide polymorphisms (SNPs), insertions and deletions (INDELs), and structural differences in the DNA itself such as copy number variants (CNVs) and chromosomal rearrangements. By studying these variations, scientists and researchers can better understand differences within a species, the causes of certain diseases, and can provide better clinical diagnoses and personalized medicine for patients.

The quality and accuracy of genome datasets is crucial to subsequent analyses and research performed on those datasets. However, imperfections in the NGS technologies used to generate these genome datasets can result in errors in both the read process itself and the read data alignment. leading to uncertainty in the output sequence(s). If an NGS machine incorrectly reads a nucleobase and records it in the read data, subsequent analysis could incorrectly identify a variant at that locus. If there are inaccuracies in the alignment of the read data, incorrect variant detection might also occur. Additionally or alternatively, an incorrect number of a repeated sequence could be detected, or an indel could be incorrectly detected or omitted. If these sources of error are left unaccounted for, false positive variant detection could lead to incorrect clinical diagnoses or the discovery of non-existent variants.

To mitigate these errors, some NGS analysis pipelines include filtering steps to detect and discard false positive variant detections. As used herein, “variant call” may be used to refer to a variant detection. Some filtering techniques employ hard filters that analyze one or more aspects of a variant call, compare it against one or more criteria, and provide a decision as to whether it is a true positive variant call or a false positive variant call. For example, if multiple read fragments aligned at a particular locus show three or more different bases, a hard filter might determine that the variant call is a false positive.

Other filtering techniques employ statistical or probabilistic models, and may involve performing statistical inferences based on one or more hand-selected variables of the variant call. A variant call might include a set of read data of DNA fragments aligned with respect to each other. Each DNA fragment read data may include metadata that specifies a confidence level of the accuracy of the read (i.e., the quality of the bases), information about the process used to read the DNA fragments, and other information. DNA sequencing experts may choose features of a variant call that they believe to differentiate true positives from false positives. Then, a statistical model (e.g., a Bayesian mixture model) may be trained using a set of labeled examples (e.g., known true variant calls and the quantitative values of the hand-selected features). Once trained, new variant calls may be provided to the statistical model, which can determine a confidence level indicative of how likely the variant call is a false positive.

False positive variant calls may be avoided or mitigated by performing more accurate read sequence alignment, and/or by improving the robustness of the variant callers themselves. Some variant callers may detect SNPs and INDELs via local de-novo assembly of haplotypes. When such a variant caller encounters a read pileup region indicative of a variant, the variant caller may attempt to reassemble or realign the sequence reads. By analyzing these realignments, these types of variant callers may evaluate the likelihood that the read pileup region contains a variant.

Many different read processes may be used to generate DNA fragment read data of a sample. Each read process may vary by read length, amplification method, materials used, and the technique used (e.g., chain termination, ligation, etc.). The nature and source of the errors of each read process may vary. Thus, the features that distinguish incorrect alignments, invalid variant calls, or false positive variant detections may differ among read processes.

Note that a “sample” may be a sample from a biological organism (e.g., a human, an animal, a plant, etc.) and/or may be a sample containing synthetic contents. For example. the sample could contain synthetic DNA (or RNA, or some other synthetic polynucleotide) created, e.g., to store information in the sequence or other characteristics of the synthetic DNA. Accordingly, the methods described herein could be applied to extract the information stored in such a sample.

### III. Terminology

### A. Next Generation Sequencing (NGS)

NGS generally refers to DNA sequencing techniques that involve sequencing multiple DNA fragments of a sample in parallel. The output data may contain nucleotide sequences for each read, which may then be assembled to form longer sequences within a gene, an entire gene, a chromosome, or a whole genome. The specific aspects of a particular NGS technique may vary depending on the sequencing instrument, vendor, and a variety of other factors. Secondary analyses may then involve aligning/assembling the reads to generate a predicted target sequence, detecting variants within the sample, etc.

An example polynucleotide (e.g., DNA) sequencing pipeline may include polynucleotide sequencing (e.g., using one or more next-generation DNA sequencers), read data alignment, and variant calling. As described herein, a “pipeline” may refer to a combination of hardware and/or software that receives an input material or data and generates a model or output data. The example pipeline receives a polynucleotide-containing sample as input, which is sequenced by polynucleotide sequencer(s) to output read data. Read data alignment occurs by receiving the raw input read data and generating aligned read data. Variant calling can then proceed by analyzing the aligned read data and outputting potential variants.

The input sample may be a biological sample (e.g., biopsy material) taken from a particular organism (e.g., a human). The sample may be isolated DNA, RNA, or some other polynucleotide and may contain individual genes, gene clusters, full chromosomes, or entire genomes. Polynucleotides of interest in a sample can include natural or artificial DNA, RNA, or other polynucleotide formed of some other type of nucleotide and/or combination of types of nucleotides. The sample may include material or DNA isolated from two or more types of cells within a particular organism. Where the sample contains RNA, it may contain multiple different isoforms of a particular RNA sequence (e.g., relating to respective different isoforms of a folded RNA, protein generated from the RNA by a ribosome or other structure(s), or some other RNA-related substance).

The polynucleotide sequencer(s) may include any scientific instrument that performs polynucleotide sequencing (e.g., DNA sequencing, RNA sequencing) autonomously or semi-autonomously. Such a polynucleotide sequencer may receive a sample as an input, carry out steps to break down and analyze the sample, and generate read data representing sequences of read fragments of the polynucleotide(s) in the sample. A polynucleotide sequencer may subject DNA (or some other polynucleotide) from the sample to fragmentation and/or ligation to produce a set of polynucleotide fragments. The fragments may then be amplified (e.g., using polymerase chain reaction (PCR)) to produce copies of each polynucleotide fragment. Then, the polynucleotide sequencer may sequence the amplified polynucleotide fragments using, for example, imaging techniques that illuminate the fragments and measure the light reflecting off them to determine the nucleotide sequence of the fragments. Those nucleotide sequence reads may then be output as read data (e.g., a text file with the nucleotide sequence and other metadata) and stored onto a storage medium.

Read data alignment can include any combination of hardware and software that receives raw polynucleotide fragment read data and generates the aligned read data. In some embodiments, the read data is aligned to a reference genome (although, one or more nucleotides or segments of nucleotides within a read fragment may differ from the reference genome). In some instances, the polynucleotide sequencer may also align the read fragments and output aligned read data.

Aligned read data may be any signal or data indicative of the read data and the manner in which each fragment in the read data is aligned. An example data format of the aligned read data is the SAM format. A SAM file is a tab-delimited text file that includes sequence alignment data and associated metadata. Other data formats may also be used (e.g., pileup format).

A variant calling method/system may be any combination of hardware and software that detects variants in the aligned read data and outputs potential variants. The variant caller may identify nucleotide variations among multiple aligned reads at a particular location on a gene (e.g., a heterozygous SNP), identify nucleotide variations between one or more aligned reads at a particular location on a gene and a reference genome (e.g., a homozygous SNP), and/or detect any other type of variation within the aligned read data. The variant caller may output data indicative of the detected variants in a variety of file formats. such as variant call format (VCF) which specifies the location (e.g., chromosome and position) of the variant, the type of variant, and other metadata.

### B. Reference Genome

As described herein, a “reference genome” may refer to polynucleotide sequencing data and/or an associated predetermined nucleotide sequence for a particular sample. This could include DNA sequences (e.g., for the genomes of plants, animals, bacteria, DNA viruses, etc.), RNA sequences (e.g., for the genomes of RNA viruses), or some other polynucleotide sequence of an organism of interest. A reference genome may also include information about the sample, such as its biopsy source, gender, species, phenotypic data, and other characterizations. A reference genome may also be referred to as a “gold standard” or “platinum” genome, indicating a high confidence of the accuracy of the determined nucleotide sequence. An example reference genome is the NA12878 sample data and genome. In examples wherein the sample contains a synthetic DNA or other synthetic polynucleotide (e.g., samples wherein containing synthetic DNA used to store information in the sequence or other characteristics of the synthetic DNA), the reference genome could be a record of a baseline, unmodified, or otherwise reference state of the synthetic DNA in the sample.

### C. Variant Types and Detection

As described herein, a genome may contain multiple chromosomes, each of which may include genes. Each gene may exist at a position on a chromosome referred to as the “gene locus.” Differences between genes (i.e., one or more variants at a particular gene locus) in different samples may be referred to as an allele. Collectively, a particular set of alleles in a sample may form the “genotype” of that sample.

Two genes, or, more generally, any nucleotide sequences that differ from each other (in terms of length, nucleotide bases, etc.) may include one or more variants. In some instances, a single sample may contain two different alleles at a particular gene locus; such variants may be referred to as “heterozygous” variants. Heterozygous variants may exist when a sample inherits one allele from one parent and a different allele from another parent; since diploid organisms (e.g., humans) inherit a copy of the same chromosome from each parent, variations likely exist between the two chromosomes. In other instances, a single sample may contain a gene that varies from a reference genome; such variants may be referred to as “homozygous” variants.

Many different types of variants may be present between two different alleles. Single nucleotide polymorphism (SNP) variants exist when two genes have different nucleotide bases at a particular location on the gene. Insertions or deletions (INDELs) exist between two genes when one gene contains a nucleotide sequence, while another gene contains a portion of that nucleotide sequence (with one or more nucleotide bases removed) and/or contains additional nucleotide bases (insertions). Structural differences can exist between two genes as well, such as duplications, inversions, and copy-number variations (CNVs).

Depending on the sensitivity and implementation of a variant caller, read data from a whole genome may include millions of potential variants. Some of these potential variants may be true variants (such as those described above), while others may be false positive detections.

### IV. Example Regional Polynucleotide Fragment Barcoding

It is desirable in a variety of applications to unambiguously determine a sequence for DNA, RNA, or some other target polynucleotide in a sample. Current NGS or other moderm sequencing techniques generate a large number of fragment read sequences from a target polynucleotide which must then be aligned or otherwise assembled into a reconstruction of the underlying sequence of the target polynucleotide. However, the presence of repeated or similar sequences at a variety of scales within natural DNA/RNA, as well as other patterns in the structure of natural DNA/RNA, make alignment of such fragment read sequences computationally expensive. Additionally, there can be ambiguity and/or inaccuracy in the alignment of the fragment read sequences (to each other and/or to an underlying reference sequence) and/or in an underlying sequence reconstructed therefrom. The systems and methods provided herein improve the process of sequencing by adding region-specific barcodes to the ends of fragments of a target polynucleotide (e.g., DNA, RNA, synthetic polynucleotides) prior to sequencing. Such region-specific barcodes indicate that fragments that include the same barcode sequence correspond to the same region within the target (e.g., the fragments were neighbors and/or correspond to respective different locations within a single contiguous range of locations within the target).

These barcode sequences can be used simplify and/or improve the accuracy of the fragment read sequence alignment process by allowing fragment read sequences bearing the same barcode to be mapped to the same region of the reconstructed sequence. This can reduce the computational cost of the alignment process by allowing the fragment read sequences to be pre-aligned via matching of the barcode sequences. Additionally, this regional information can improve the accuracy of the alignment/sequencing by providing additional distal information about the association between fragment sequences. Such alignment and/or sequencing processes can include using barcode sequences that are present in fragment read sequences to identify the fragment read sequences as belonging to the same target polynucleotide (e.g., the same one of a set of two diploid chromosomes, the same isoform of multiple isoforms of RNA transcoded from the same gene), to align the fragment read sequences (e.g., to align them in a manner that obviates ambiguities regarding the presence of an indel, a number of repeat sequences, or some other ambiguity that would be present in the absence of the barcode sequences), and/or to facilitate and/or improve some other aspect of sequencing.

The methods described herein form such regionally-specific barcode sequences onto the ends of fragments of a target polynucleotide by fragmenting the target polynucleotide while tethering neighboring fragments of the target polynucleotide together. This can be done, e.g. by ligating polynucleotide ‘end caps’ onto the newly-formed ends of neighboring fragments, with the end caps tethered together via a length of polyethylene glycol or some other linking agent. The fragmentation allows the regionally-specific barcode sequences to be ligated onto the fragments of the target polynucleotide. Tethering neighboring fragments together allows those fragments to be subjected to ligation with the same sequence of shorter barcode sub-sequences, such that the same complete regionally-specific barcode sequence is sequentially ‘grown’ onto each of the tethered-together fragments.

Different regionally-specific barcode sequences can be grown onto different sets of tethered-together fragments, e.g., different chromosomes, different isoforms of an RNA transcribed from the same gene, different portions of a single polynucleotide that is fragmented before and/or after the tethered fragmentation process describe herein. This can be done quickly and efficiently by employing a repeated split-pool process. A sample containing a mixture of the different sets of tethered-together fragments is split into a number of different sub-samples. Fragments in each of the sub-samples are then ligated with a sample-specific barcode sub-sequence (e.g., fragments in a first sub-sample have a first sub-sequence ligated onto their end(s), and fragments in a second sub-sample have a second, different sub-sequence ligated onto their end(s)). The different sub-samples are then pooled, and the steps of separation of the pooled sample into separate sub-samples and ligation with sub-sample-specific barcode sub-sequences are repeated. Thus, fragments of a particular set of tethered-together fragments will exhibit the same complete regionally-specific barcode sequence that is composed of the sub sample-specific sub-sequences to which the particular set was exposed, ordered according to the ordering of exposure of the particular set to the various sub-samples of which it was a part. The number of sub-samples per split-pool cycle, the number of repetitions of the split-pool cycle, the specifics of the sub-sample-specific barcode sub-sequences and/or of their ligation to the fragments, or other properties of the repeated split-pool process can be selected to reduce the likelihood that any two different sets of tethered-together fragments will exhibit the same regionally-specific barcode sequence by reducing the likelihood that any two different sets of tethered-together fragments share that same ‘path’ from sub-sample to sub-sample across the entire repeated split-pool process. Such a repeated split-pool process thus allows the growth of a large number of regionally-specific barcode sequences in a manner that is quick and extremely low-cost.

Once the regionally-specific barcode sequences have been ‘grown’ onto the fragments, the linkers holding the fragments together can be severed (e.g., by using click chemistry or some other means to reliably decouple the neighboring fragments from each other without significantly negatively affecting the fragments and/or the regional barcodes formed thereon or significantly negatively affecting the ability to sequence such fragments). To allow for the formation of ‘sub-regional’ barcodes, additional fragmentation (without tethering) could be performed following one or more of the split-pool ligation cycles described herein, followed by one or more additional split-pool cycles. This would result in fragments from a particular region exhibiting the same regional barcode sequence up until the cycle prior to the additional fragmentation. The regional barcode sequences for cycle(s) subsequent to the additional fragmentation would differ between fragments from the different sub-regions (with the sub-regions being distinguished by the location(s) of the additional fragmentation).

Fragmenting a target polynucleotide while keeping neighboring fragments tethered together can be accomplished by ligating polynucleotide ‘end caps’ onto the newly-formed ends of neighboring fragments, with the end caps tethered together via linker The linker could be a specified length of a polymer (e.g., polyethylene glycol) or other long, flexible chemical substance that does not significantly impede the ligation of additional barcode sequences onto the fragments. The linker could be coupled to the end caps via click chemistry or via some other chemical means that facilitates reliably severing the linkers from the end caps without significantly negatively affecting the fragments and/or the end caps or regional barcodes ligated thereto. This could include coupling the ends of the linker to a modified nucleotide of the end caps (e.g., to i5OctdU nucleotides in the end caps).

The end caps can include specified sequences or other structure to facilitate ligation of barcode sub-sequences specifically to the end caps. This could include the end caps being composed of dsDNA, with one of the strands of the DNA being longer than the other by a specified recognition sequence. The identity and length of that sequence could then be leveraged to specifically ligate a regionally-specific barcode sequence or sub-sequence onto the end caps. For example, the end caps could be dsDNA, one strand of which extends beyond the other by a specified 4 bp sequence that can be recognized by a T7 ligase used to ligate a barcode sub-sequence thereto. Such a barcode sub-sequence could, itself, include a terminal recognition sequence to facilitate ligation of further barcode sub-sequences thereto. Such terminal recognition sequences could differ from sequence to sequence. This could be done in order to improve the specificity of ligation between cycles of a repeated split-pool process, thereby facilitating failure detection of sub-barcode ligation sequence in one or more of the cycles.

Fragmentation of the target polynucleotide and ligation of a pair of tethered-together end caps to the neighboring ends of the newly-formed fragments of the target polynucleotide could include a variety of substances and/or processes. In some examples, a probe could be added to a sample containing the target polynucleotide. Such a probe could include first and second payload polynucleotides (which, when ligated onto fragments of the target polynucleotide, will become the ‘end caps’) tethered together via a linker. Such a probe could also include an insertion vector that configured to achieve fragmentation of the target polynucleotide and ligation of the payload polynucleotides to the ends of the fragments created by the fragmentation. Such an insertion vector could include a single protein, DNA, RNA, or other substance to perform both of these functions, or could include elements (e.g., ligases) for ligating the payload polynucleotides onto the ends of the fragments and separate elements (e.g., restriction enzymes) for fragmenting the target polynucleotide. In some examples, two instances of a fragmentation and/or ligation agent could be included in the probe, with each instance associated with a respective one of the payload polynucleotides. In some examples, the payload polynucleotides could include specified sequences (e.g., ‘mosaic’ sequences) to facilitate the specific association of the payload polynucleotides with the insertion vector(s) (e.g., a 19 bp mosaic sequence specified to facilitate association with a corresponding Tn5 transposase).

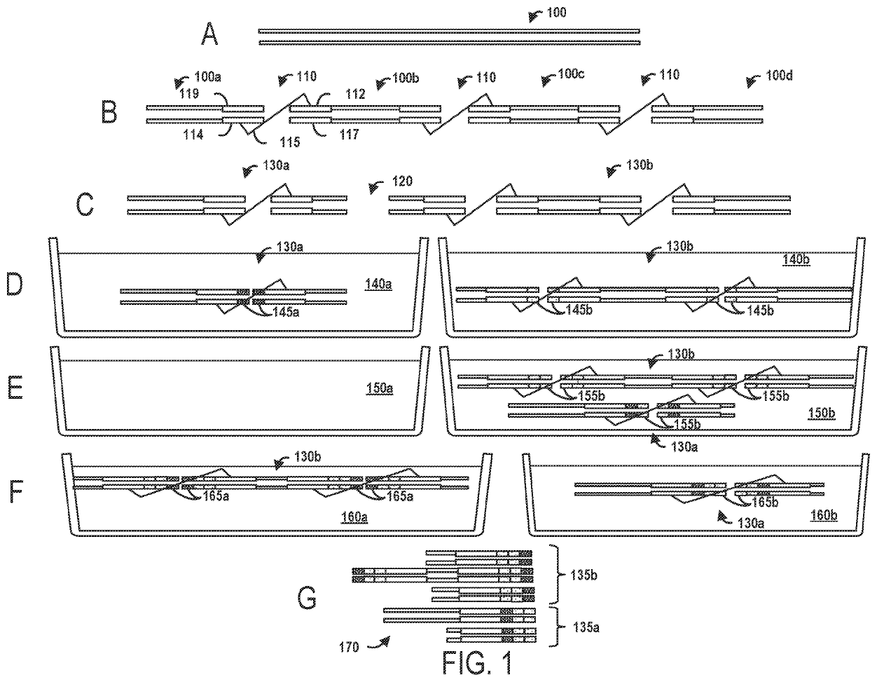

FIG. 1 illustrates aspect of an example process for creating regionally-specific barcodes on fragments of a target polynucleotide such that fragments from the same contiguous region of the target polynucleotide exhibit the same regionally-specific barcode while fragments that are not from that region exhibit different regionally-specific barcode(s). Step “A” illustrates the target polynucleotide 100. The target polynucleotide 100 is a length of dsDNA having a sense strand (upper strand in Step A) and a complementary anti-sense strand (lower strand in Step A. Note that the methods described herein could be adapted, with appropriate modification, to target polynucleotides that are composed of ssDNA, RNA, some other natural or artificial nucleobases and/or some combination thereof. For example, the target polynucleotide could be a cDNA generated from an RNA of interest. The target polynucleotide could be the entirety of a chromosome (e.g, a particular chromosome of a pair of chromosomes), mRNA (e.g., a particular isoform of mRNA transcribed from a particular locus or gene), or other naturally-terminated polynucleotide or could be a specified portion thereof, e.g., a specified gene, set of genes, allele, or other specified locus within a larger polynucleotide. Additionally or alternatively, the target polynucleotide could be a randomly-terminated fragment of such a naturally-terminated polynucleotide or portion thereof. For example, the target polynucleotide 100 could be a randomly-terminated fragment of a chromosome.

The target polynucleotide 100 could be isolated and/or purified such that it is the only polynucleotide present in a sample. Alternatively, the target polynucleotide 100 could be one of a plurality of different polynucleotides (e.g., other chromosome or fragments thereof, other isoforms of RNA corresponding to the same locus or gene) present in a sample. The target polynucleotide 100 could be amplified (e.g., via a process of polymerase chain reaction (PCR) or some other amplification process), fragmented (e.g., by the application of restriction enzymes), ligated, and/or processed in some other manner.

Step “B’ of FIG. 1 illustrates the target polynucleotide 100 after having been fragmented into a number of fragments 100a, 100b, 100c, 100d and with neighboring fragments (e.g., 100a and 100b) being coupled together via the insertion of tethered dimers 110. Each tethered dimer 110 includes first and second “end caps” composed of payload polynucleotides that match the target polynucleotide 100 (dsDNA in FIG. 1) that are tethered together via a linker. This is illustrated by a first end cap composed of a first payload polynucleotide 114 that is ligated to the 5′ end of the anti-sense strand of a first fragment 100a and that is tethered, via a linker 115 (e.g., a length of polyethylene glycol) to a second payload polynucleotide 112 that is ligated to the 5′ end of the sense strand of a second fragment 100b. Also included are a third payload polynucleotide 119 that is at least partially complementary to the first payload polynucleotide 114 and that is ligated to the 3′ end of the sense strand of the first fragment 100a and a fourth payload polynucleotide 117 that is at least partially complementary to the second payload polynucleotide 112 and that is ligated to the 3′ end of the anti-sense strand of the second fragment 100b.

The end caps can be inserted into the target polynucleotide 100 by, e.g, introducing probes containing the tethered-together end caps into a sample that contains the target polynucleotide 100 and/or fragments or copies thereof. Such probes can include an insertion vector (e.g., CRISPR-Cas9, Tn5 transposase) to insert the payload polynucleotides into the target polynucleotide 100 and/or to fragment the target polynucleotide 100. The probes could include other elements or features. In some examples, the probes could be configured to insert the barcodes into specified location(s)s of the target polynucleotide 100 (e.g., to facilitate sequencing of a specific locus within the target polynucleotide 100, to increase the likelihood that the barcode is inserted into a repeating region or other region especial interest).

Step “C” of FIG. 1 shows the target polynucleotide 100 after having been further fragmented (at location 120). This results in the information of a first set 130a of tethered-together fragments of the target polynucleotide and a second set 130b of tethered-together fragments of the target polynucleotide 100. The first set 130a includes the first fragment 100a and a portion of the second fragment 100b while the second set 130b includes the remainder of the second fragment 100b and the third 100c and fourth 100d fragments. The fragments within a set being tethered together makes it likely that, if a sample containing the sets 130a, 130b, etc. is separated into sub-samples, all of the fragments in a single set of tethered-together fragments will be separated into the same sub-sample, even through multiple instances of splitting and/or pooling of such samples or sub-samples. Accordingly, all of the fragments in a single set of tethered-together fragments will be exposed to the same environmental conditions of such samples or sub-samples, and thus can have ‘grown’ thereon the same regionally-specific barcode sequence.

Step “D” of FIG. 1 shows the result of such a separation into first 140a and second 140b sub-samples. The first set 130a has been separated into the first 140a sub-sample and the second set 130b has been separated into the second 140b sub-sample. Each of the sub-samples 140a, 140b contains substances (e.g., instances of T7 ligase, T4 ligase, or some other ligating substance coupled to barcode polynucleotide sequences) such that end caps present in the first sub-sample 140a have ligated thereon first barcode sequences 145a and such that end caps present in the second sub-sample 140b have ligated thereon second barcode sequences 145b.

The samples 140a, 140b can then be pooled together and separated into further sub-samples, thereby ‘growing’ unique and regionally-specific barcode sequences onto all of the fragments in every set (e.g., 130a, 130b) of tethered-together fragments of the target polynucleotide 100. Step “E” of FIG. 1 shows a second separation, of a pooled sample comprising the first 140a and second 140b sub-samples, into third 150a and fourth 150b sub-samples. Both first set 130a and second set 130b have, by chance, been separated into the second 140b sub-sample. Each of the sub-samples 150a, 150b contains substances such that end caps present in the third sub-sample 150a have ligated thereon third barcode sequences (not shown) and such that end caps present in the fourth sub-sample 150b have ligated thereon fifth barcode sequences 155b. Step “F” of FIG. 1 shows a third separation, of a pooled sample comprising the third 150a and fourth 150b sub-samples, into fifth 160a and sixth 160b sub-samples. By chance, the first set 130a has been separated into the sixth 160b sub-sample and the second set 130b has been separated into the fifth 160a sub-sample. Each of the sub-samples 160a, 160b contains substances such that end caps present in the fifth sub-sample 160a have ligated thereon fifth barcode sequences 165a and such that end caps present in the sixth sub-sample 160b have ligated thereon sixth barcode sequences 165b.

Note that the barcode sequences added as part of each cycle of splitting and pooling could be the same as sequences added during prior/subsequent cycles, or different. The barcode sequences differing from cycle to cycle could assist in preventing and/or facilitating the detection of instances where a particular fragment failed to be extended as expected from exposure to one or more of the sub-samples 140a, 140b, 150a, 150b, 160a, 160b. These differences could include the barcode sequences terminating with recognition sequences (e.g., 4 bp recognition sequences) to facilitate increased specificity in the binding of the barcodes to the fragments.

The final sub-samples can then be pooled and the linkers (e.g., example linker 115) decoupled from their corresponding end caps (e.g., via click chemistry), thereby resulting in a plurality of different fragments 170 of the target polynucleotide 100. Each of the fragments 170 has been extended to include a barcode sequence that represents the ‘path’ of the fragment through the various sub-samples 140a, 140b, 150a, 150b, 160a, 160b. Thus, a first subset 135a of the fragments 170, which were part of the first set 130a of tethered-together fragments, end in a first regionally-specific barcode (the first 145a, fourth 155b, and sixth 165b barcode sequences, in order) and a second subset 135b of the fragments 170, which were part of the second set 130b of tethered-together fragments, end in a second, different regionally-specific barcode (the second 145b, fourth 155b, and fifth 165a barcode sequences, in order).

These fragments 170 can then be sequenced to generate corresponding read fragment sequences and the contents of the regionally-specific barcode in each of the read fragment sequences used to associate the read fragment sequences together by region. This association can be used to speed and/or reduce the computational cost of alignment of the read fragment sequences by allowing the read fragment sequences to be ‘pre-aligned’ using their association according to the regionally-specific barcode sequences. Additionally or alternatively, this association can also be used to generate higher-accuracy alignments by leveraging the distal sequence information represented by the regional association between the read fragment sequences.

Note that the sets 130a, 130b of tethered-together fragments being generated from the same target polynucleotide 100 is intended as a non-limiting example embodiment. In practice, such sets of tethered-together polynucleotide fragments could be from different source polynucleotides (e.g., different chromosomes, mRNA transcribed from different genes, different isoforms of mRNA transcribed from the same gene). Further, the further fragmentation of the target polynucleotide 100 following insertion of the tethered pairs of end caps 110 (shown in Step C of FIG. 1) is intended as a non-limiting example embodiment. In practice, such fragmentation could alternatively or additionally occur prior to insertion of tethered pairs of end caps into one or more target polynucleotides.

In some examples, additional fragmentation could occur following one or more split-pool cycles and prior to one or more additional split-pool cycles. This could be done to create sub-sets of tethered-together fragments that exhibit regionally-specific barcodes that are partially the same across all of the fragments of the sub-sets (thus indicating the larger region of a target polynucleotide from which all of the fragments came) but that are also partially different from sub-set to sub-set (thus indicating the distinct sub-regions, within the larger region, of the target polynucleotide from which all of the fragments of each of the distinct sub-sets came). This combined regional and sub-regional barcoding can provide further benefits with respect to the speed and/or computational cost of aligning the barcoded fragments, increase accuracy of alignment of the fragments and/or reconstruction of the sequence of a target polynucleotide, or other benefits.

Note that the number of sub-samples per split-pool cycle illustrated in FIG. 1 (two) and the number of repetitions of the split-pool barcode ligation process illustrated in FIG. 1 (three) are intended as non-limiting examples for the purpose of illustration. More or fewer split-pool barcode ligation cycles could be employed. and more sub-samples could be generated as part of each of the split-pool barcode ligation cycles. The number of cycles, number of samples per cycle, occurrence and timing of additional fragmentation steps within the set of cycles, or other properties of a repeated split-pool barcode ligation cycle process as described herein could be specified in order to reduce a cost or experimental complexity of the process, to provide for increased likelihood that no two different sets of tethered-together fragments exhibit the same regionally-specific barcode, to reduce the computational cost of alignment or reconstruction of a target polynucleotide sequence, to increase an accuracy of reconstruction of a target polynucleotide sequence, or to adjust some other benefit or factor related to the process.

The systems and methods described herein include inserting paired, tethered-together payload polynucleotides (alternatively referred to as ‘end caps’) into a target polynucleotide in order to facilitate the ‘growth’ of regionally-specific barcode sequences thereon, thereby improving the cost, accuracy, or other aspects of sequencing the target polynucleotide and/or selected portions thereof. A variety of substances and methods can be employed to fragment a target polynucleotide and to ligate a pair of tethered-together payload polynucleotides onto the adjacent ends of the newly-formed fragments of the target polynucleotide. In some examples, this can include creating a plurality of probes, each probe including an insertion vector, two payload polynucleotides (which will become the ‘end caps,’ once ligated onto the ends of neighboring fragments formed from a target polynucleotide), and a linker that is coupled to the payload polynucleotides and that will keep, and the fragments they are ligated onto, coupled together as part of a set of tethered-together fragments of the target polynucleotide. The insertion vector is one or more structures (e.g., a protein, DNA, RNA, and/or other substances or structures) configured to fragment the target polynucleotide and to attach the payload polynucleotides onto the neighboring ends of the newly-formed fragments of the target polynucleotide. The payload polynucleotides may be dsDNA, ssDNA, RNA, or some other variety of polynucleotide, usually corresponding to the structure of the target polynucleotide.

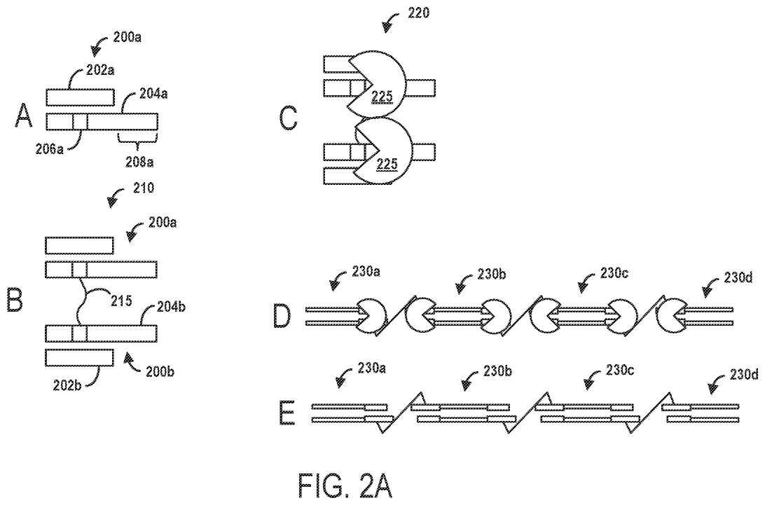

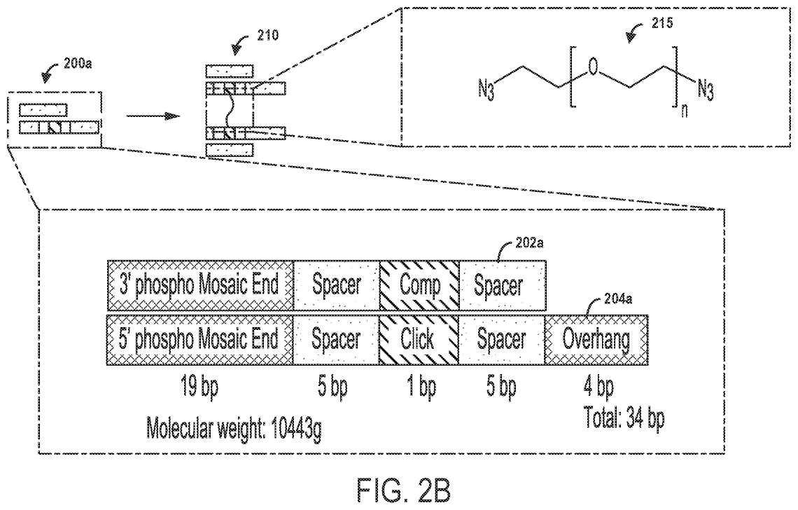

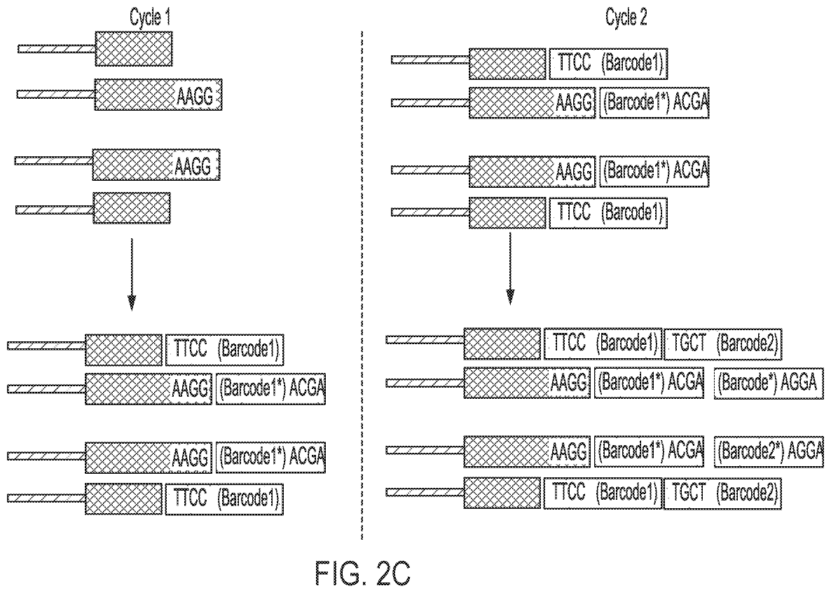

FIG. 2A illustrates, by way of example, aspects of such a probe and steps for creating such a probe and for inserting it into a target polynucleotide. In a first step (Step “A” of FIG. 2A) a first dsDNA end cap 200a is provided. The first end cap 200a includes a first payload polynucleotide 204a and a second payload polynucleotide 202a that are at least partially complementary and that are associated with each other as dsDNA. The first end cap 200a could be created via a variety of processes, e.g., via a tailored oligonucleotide synthesis according to a specified sequence followed by amplification of the synthesized oligonucleotide to generate sufficient quantities of the first end cap 200a.

As shown, the first payload polynucleotide 204a extends beyond the second payload polynucleotide 202a by a few base pairs of an overhang 208a. The overhang 208a could have a length and/or sequence specified to facilitate ligation of barcode sequences onto the first end cap 200a following attachment of the first end cap 200a onto the end of a fragment of a target polynucleotide. The overhang 208a could include a recognition sequence to facilitate recognition of the end cap 200a by a ligase or other elements used to ligate a barcode sequence onto the end cap 200a. Additionally, the location of the overhang 208a relative to the direction of the first payload polynucleotide 204a (i.e., 3′ end vs. 5′ end) could be selected according to the ligase or other elements used to ligate a barcode sequence onto the end cap 200a. For example, the overhang 208a could be 4 bp long and located on the 3′ end of the first payload polynucleotide 204a so as to facilitate the use of T7 ligase (or some other appropriate attachment agent, e.g., T4 ligase) to ligate additional barcode sequences onto the first end cap 200a.

The first payload polynucleotide 204a also includes an attachment site 206a via which the first payload polynucleotide 204a can be coupled to a linker. This could be a modified polynucleotide or some other element to facilitate coupling the first payload polynucleotide 204a to the linker in such a manner that the linker can later be reliably severed from the first payload polynucleotide 204a without negatively affecting the first end cap 200a and/or any target polynucleotide fragment, barcode sequence(s), or other polynucleotides of interest attached thereto.

In Step “B” of FIG. 2A, a second end cap 200b (which may be identical to the first end cap 200a and thus created via the same process(es) used to create the first end cap 200a) is tethered to the first end cap 200a by a linker 215, thereby creating a tethered dimer 210. The second end cap 200b includes a third payload polynucleotide 204b that is associated with a fourth payload polynucleotide 202b as dsDNA. The linker 215 could be any long, flexible chemical or other substance, e.g., a length of polyethylene glycol. The length of the linker 215 could be specified to allow additional barcode or other sequences to be ligated onto the end caps 200a, 200b after their attachment onto fragments of a target polynucleotide while also reducing the risk that the fragments are mechanically or otherwise separated from each other unintentionally (e.g., due to shear in a sample during separation into sub-samples or some other sample handling process). For example, the linker 215 could be a length of polyethylene glycol or some other polymer comprising between 40 and 125 monomer subunits.

A variety of methods for coupling the linker 215 to the attachment sites (e.g., 206a) of the first 204a and third 204b payload polynucleotides. This could include coupling the linker 215 to a modified nucleotide or other element(s) of the attachment sites in a manner such that the linker 215 can later be reliably decoupled from the end caps 200a, 200b without significantly negatively affecting the end caps and/or any target polynucleotide fragment, barcode sequence(s), or other polynucleotides of interest attached thereto. For example. the linker 215 could be coupled to the end caps 200a, 200b such that it can later be decoupled using “click” chemistry methods or some other methods that result in highly reliable and specific decoupling of the linker 215 while minimally interfering with the end caps, target polynucleotide fragments, barcode sequence(s), or other polynucleotides of interest (e.g., without producing highly reactive byproducts).

In a particular example, the attachment sites could include a nucleotide that has been modified to include an extension that terminates in an alkyne group (e.g., 5-Octadiynyl dU, or “i5OctdU”). Then copper(I)-catalyzed azide-alkyne cycloaddition or some other click chemistry reaction could be used to couple chains of polyethylene glycol or some other linking agent to the modified nucleotide. For example, a mixture of CuSO4 (or some other source of copper) and tris-hydroxypropyltriazolylmethylamine (THPTA) could be added to a phosphate buffered saline mixture that contains the end caps 200a/200b and the polyethylene glycol chains. Sodium ascorbate or some other reducing agent can then be added to drive the “click” reaction, coupling the polyethylene glycol chains (or other linking agent) to the end caps.

In Step “C” of FIG. 2A, two insertion vectors 225 have been associated with the end caps 200a, 200b thereby forming a tethered dimer probe 220 that can be used to fragment a target polynucleotide and to attach the end caps 200a, 200b to respective ends of the newly-formed fragments of the target polynucleotide. The insertion vectors 225 could include CRISPR-Cas9, CRISPR-Cas12a, CRISPR associated with some other protein or complex of proteins, Tn5 transposase, Tn7 transposase, some other transposase, or some other insertion vector that can act to insert one or more payload polynucleotides into a target polynucleotide and/or to ligate one or more payload polynucleotides onto the end of a fragment of a target polynucleotide. The insertion vectors 225 could fragment the payload polynucleotide at random locations within the target polynucleotide and/or at specified locations within the target polynucleotide (e.g., at specified locations within the target polynucleotide that complement a guide RNA (gRNA) of the insertion vector). If the insertion vector is configured to insert the payload at a specified location(s), the location(s) could be specified to target locations of particular interest within the target polynucleotide, e.g., locations proximate SNPs, trinucleotide repeats, indels, or other variants of relevance to a particular disease or disorder. In some examples, one or more of the payload polynucleotides 202a, 204a, 202b, 204b could include specified sequences (e.g., “mosaic” sequences) to facilitate association with the insertion vectors 225.

Step “D” of FIG. 2A shows a target polynucleotide fragmented by insertion vectors 225 of a number of instances of the probe 220 into a number of sequential fragments 230a, 230b, 230c, 230d. Each neighboring pair of fragments (230a and 230b, 230b and 230c, 230c and 230d) is tethered together via the linker and end caps of the probe instance that fragmented the neighboring fragments apart and that ligated the end caps to the newly-formed ends of those neighboring fragments. The insertion vectors 225 can then be removed from the set of tethered-together fragments 230a, 230b, 230c, 230d and additional steps may be performed. These steps could include washing the sample to remove the insertion vectors or other substances from the sample, further fragmenting the target polynucleotide (e.g., using restriction enzymes, mechanical fragmentation, etc.), performing nick repair, annealing, or other processes to ensure the integrity of the set of tethered-together fragments, or some other process(es). The set of tethered-together fragments 230a, 230b, 230c, 230d following the performance of such processes is depicted in Step “E” of FIG. 2A.

FIG. 2B illustrates details of a particular example of a tethered dimer 210 that includes two dsDNA end caps 200a, 200b tethered together via a linker 215. FIG. 2B also illustrates details of the linker 215. Note that these details are intended as non-limiting examples of linkers and of end caps of a tethered dimer as described elsewhere herein.

As shown in the inset, the end cap 200a has a molecular weight of 10443 g and comprises a 34 bp first strand 204a and a 30 bp second strand 202a that are associated with each other as dsDNA.

The first strand 204a extends beyond the second strand 202a at the 3′ end of the first strand 204a by a 4 bp overhang sequence (“Overhang”). This overhang sequence can be used as a recognition sequence to facilitate reliable and specific ligation of first-stage barcode sequences onto the end cap 200a. Such first-stage barcode sequences could terminate in their own overhang recognition sequences. The end cap overhang sequence and the first-stage barcode overhang sequences could differ, so as to improve the specificity of ligation of second-stage barcode sequences and avoid ligation of such second-stage barcode sequences to the end cap 200a in instances where no first-stage barcode sequence was ligated onto the end cap. This can be done to ensure that failures in the regionally-specific barcode formation process do not go undetected, thus leading to potential ambiguity in barcode identification and use to align target polynucleotide fragments. A 4 bp overhang recognition sequence was found to afford sufficient specificity to allow for multiple split-pool cycles of barcode ligation to occur while ensuring significant specificity of ligation from one cycle to the next.

The first strand 204a and second strand 202a include 19 bp mosaic sequences (“3′ phospho Mosaic End” and “5′ phospho Mosaic End”) specified to facilitate association of a Tn5 transposase or other insertion vector. The content of these mosaic sequences could be specified to comport with a selected insertion vector (e.g., Tn5 transposase, a CRISPR complex) and/or the insertion vector could be modified to associate with the mosaic sequences. Such mosaic or other insertion vector recognition sites could have different lengths to accommodate different insertion vectors. Depending on the particulars of the insertion vector. such mosaic sequences may or may not be ligated, in whole or in part, onto the end of fragments of a target polynucleotide.

The first strand 204a includes a modified nucleotide (“Click”) that can be coupled to a linker agent using “click” chemistry or some other suitable chemistry that permits reliable and specific release of a linker while reducing the likelihood that the release chemistry causes unwanted effects (e.g., polynucleotide fragmentation, methylation, etc.) on the end caps or target polynucleotide fragments, barcode sequences, or other polynucleotides attached thereto. The modified nucleotide is flanked by two 5 bp spacer sequences (“Spacer”). The length and/or content of these spacer sequences could be specified to comport with requirements of the insertion vector, of a ligation agent used to ligate barcodes onto the end cap 200a, with a chemistry used to attached a linker to the modified nucleotide. or satisfy some other criterion The second strand 202a includes a complement nucleotide (“Comp”) to the modified nucleotide. The complement nucleotide is flanked by two 5 bp spacer sequences (“Spacer”) that are complementary to the spacer sequences of the first strand 204a.

As shown in FIG. 2B, the linker 215 can be a chain of polyethylene glycol having a length (“n”) between, e.g., 40 and 125 monomer subunits. However, alternative polymers or other long, flexible chemical elements could be used. Prior to coupling to the end caps 200a, 200b, the linker 215 can be terminated in amines (as shown in the inset) to facilitate coupling via copper(I)-catalyzed azide-alkyne cycloaddition or some other chemical reaction.

As noted above, the end caps of tethered dimers as described herein could terminate in recognition sequences (e.g., recognition sequences of a first strand of dsDNA that overhang their complement strand of dsDNA by a specified amount) to facilitate specific ligation of barcode sequences onto the end caps. Those barcodes could, themselves, terminate in recognition sequences to facilitate specific ligation of further barcode sequences. The recognition sequences of the end caps and the barcodes could be the same. However, in such an example, an end cap to which a first barcode was not ligated could then have a second barcode ligated onto itself, or a first instance of a first barcode could have another instance of the first barcode ligated thereon. These circumstances could lead to ambiguous and/or varying barcodes across the fragments of a set of tethered-together target polynucleotide fragments. Instead, the recognition sequences could vary from one cycle of ligation to the next, reducing variability in the barcodes across the fragments of a set of tethered-together target polynucleotide fragments and facilitating the detection and/or rejection of fragments onto which erroneous barcodes are formed.

The top of the left pane of FIG. 2C depicts two dsDNA fragments of a target polynucleotide that are tethered together via dsDNA end caps (cross-hatched portions) that are coupled to each other via a linker (not shown). Each of the end caps ends in a recognition sequence “AAGG.” A regionally-specific barcode can be grown on the end caps, using a repeated split-pool process as described herein, by sequentially ligating shorter barcode sequences onto the end caps. The bottom of the left pane of FIG. 2C depicts the specific ligation of first dsDNA barcodes onto the end caps. As shown, the first barcodes include a first strand whose contents include a complement sequence “TTCC” to the recognition sequence “AAGG” of the end caps, as well as a first barcode sequence (“Barcode1”). The first barcodes also include a second strand whose contents include a complement to the first barcode sequence (“Barcode1*”) and a second recognition sequence “ACGA.”

This second recognition sequence can be targeted to specifically ligate second dsDNA barcodes onto the first dsDNA barcodes. The top of the right pane of FIG. 2C depicts the target polynucleotide fragments, end caps, and first dsDNA barcodes prior to ligation of such second dsDNA barcodes. The bottom of the right pane of FIG. 2C depicts the specific ligation of the second dsDNA barcodes onto the first dsDNA barcodes. As shown, the second barcodes include a first strand whose contents include a complement sequence “TGCT” to the recognition sequence “ACGA” of the first barcodes, as well as a second barcode sequence (“Barcode2”). The second barcodes also include a second strand whose contents include a complement to the second barcode sequence (“Barcode2*”) and a third recognition sequence “AGGA.” This third recognition sequence can be targeted for ligation of a third round of dsDNA barcodes.

Different dsDNA barcodes, corresponding to different sub-samples of a single split-pool ligation cycle. will begin with the same complement sequences and terminate with the same recognition sequences, to facilitate sequential ligation of additional barcode sequences from one split-pool cycle to the next. So, for example, the first dsDNA barcode depicted in FIG. 2C could be provided in a first sub-sample of a first split-pool cycle while a third dsDNA barcode is provided in a second sub-sample of the first split-pool cycle. The third dsDNA barcode could have a first strand whose contents include a complement sequence “TTCC” to the recognition sequence “AAGG” of the end caps, as well as a third barcode sequence. The third barcodes could also include a second strand whose contents include a complement to the third barcode sequence and the second recognition sequence “ACGA,” making the third dsDNA barcodes able to be ligated onto the end caps in the cycle, while also permitting ligation onto the third barcodes by the second barcode or by some other dsDNA barcode of the second split-pool cycle.

Note that the use of such recognition sequences, which vary from cycle to cycle of a sequential process of ligation of barcode sequences to form a regionally-specific barcode sequence, in the dsDNA context is intended as a non-limiting example. The use of cycle-specific recognition sequences could be adapted to an ssDNA context, an RNA context, or some other polynucleotide context.

Additionally, the use of 4 bp recognition sequences is intended as a non-limiting example; longer or shorter recognition sequences could be employed according to the constraints of a particular application. Experimental data has shown that 4 bp recognition sequences, when implemented as overhangs of dsDNA barcodes that are ligated-to using T7 ligase, are able to facilitate cycle-specific ligation of such barcodes as part of a repeated split-pool process or other repeated barcode ligation process as described herein.

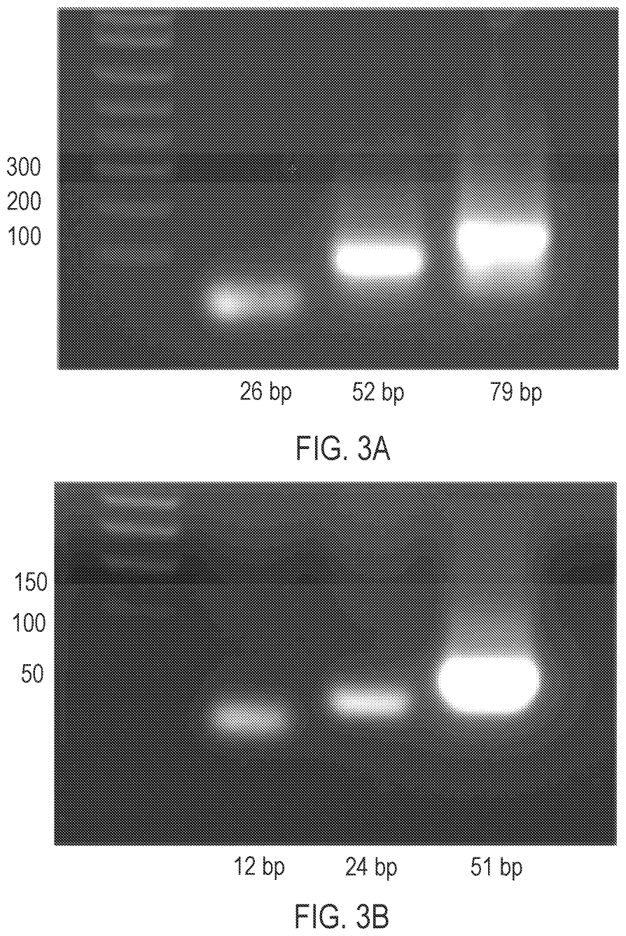

In a first experiment, first, second, and third dsDNA barcodes were sequentially ligated together. The first dsDNA barcode comprised two 26 bp strands, one strand beginning with a “GATC” complement overhang sequence and a second strand terminating with an “AGTT” recognition overhang sequence. The second dsDNA barcode comprised two 26 bp strands, one strand beginning with a “TCAA” complement overhang sequence (complementary to the recognition sequence of the first barcode) and a second strand terminating with an “GCTA” recognition overhang sequence. The third dsDNA barcode comprised a first 29 bp strand beginning with a “CGAT” complement overhang sequence (complementary to the recognition sequence of the second barcode) and a second 25 bp strand. A first sample contained only the first barcode, a second sample was generated by using T7 ligase to ligate the second barcode onto the first barcode, and a third sample was generated by using T7 ligase to ligate the second barcode onto the first barcode and then to ligate the third barcode onto the second barcode. The specificity of this sequential ligation process, using 4 bp recognition sequences, was confirmed via gel electrophoresis of each of the three samples. FIG. 3A shows the result of that gel electrophoresis, and depicts bands at the expected 26 bp, 52 bp, and 79 bp locations for the contents of the first, second. and third samples, respectively.

In a second experiment. nine different dsDNA barcodes, organized into three different, sequential split-pool ligation cycles, were used to generate three different pooled samples of barcodes. The three different dsDNA barcodes of the first cycle each comprised two 12 bp strands, one strand beginning with a “GATC” complement overhang sequence and a second strand terminating with an “AGTT” recognition overhang sequence. The central ‘barcode’ sequences differed between the three first-cycle barcodes. The three different dsDNA barcodes of the second cycle each comprised two 12 bp strands, one strand beginning with a “TCAA” complement overhang sequence (complementary to the recognition sequence of the barcodes of the first cycle) and a second strand terminating with an “GCTA” recognition overhang sequence. The central ‘barcode’ sequences differed between the three second-cycle barcodes. The three different dsDNA barcodes of the third cycle each comprised a first 29 bp strand beginning with a “CGAT” complement overhang sequence (complementary to the recognition sequence of the barcodes of the second cycle) and a second 25 bp strand. The terminal ‘barcode’ sequences differed between the three third-cycle barcodes.

A first sample was created by pooling samples individually containing one of the first-cycle barcodes. A second sample was generated by splitting a portion of the first sample into three sub-samples, using T7 ligase to ligate one of the three second-cycle barcodes onto the first-cycle barcodes in each of the three sub-samples, and then pooling the three sub-samples together into the second sample. A third sample was generated by splitting a portion of the second sample into three sub-samples, using T7 ligase to ligate one of the three third-cycle barcodes onto the second-cycle barcodes in each of the three sub-samples, and then pooling the three sub-samples together into the second sample. The specificity of this sequential ligation process, using 4 bp recognition sequences in the split-pool context, was confirmed via gel electrophoresis of each of the three samples. FIG. 3B shows the result of that gel electrophoresis, and depicts bands at the expected 12 bp, 24 bp, and 51 bp locations for the contents of the first, second, and third samples, respectively.

In some examples, a “final” split-pool cycle could ligate a final dsDNA (or otherwise configured) barcode sequence that also includes primer sequences, recognition sequences for ligation onto oligonucleotides of a solid support, or some other additional contents to facilitate further process steps. Additionally or alternatively, such primer sequences, recognition sequences for ligation onto oligonucleotides of a solid support, or other additional contents could be ligated onto a growing polynucleotide fragment without also including a sub-sample-specific barcode sequence.

### V. Example Polypeptide Barcode Generation and Sequencing

As described above, the insertion of polynucleotide barcodes into a target polynucleotide can provide a variety of benefits with respect to determining a sequence of the target polynucleotide. These benefits generally relate to the ability to ‘mark’ fragments from the same region of a single source polynucleotide with regionally-specific barcodes that are indicative of that source region, thereby providing additional sequencing information that allows the corresponding fragment read sequences to be more easily and/or more accurately associated together. These processes can also be adapted to provide improvements in the field of polypeptide sequencing.

Such adaptation includes marking individual polypeptide molecules multiple times with a polynucleotide probe (e.g., a probe that includes dsDNA) that can then be expanded, via the processes described above (e.g., repeated split-pool cycles of ligation of barcodes to the probes), to exhibit a regionally-specific barcode. Once marked in this manner, the polypeptide molecules can be fragmented and the fragments can then be sequenced in parallel with their associated polynucleotide barcodes. The regionally-specific polynucleotide barcode sequences can then be used to associate the polypeptide sequences together (into the same instance of the same or different polypeptide, or into respective differently-barcoded regions of the same instance of a polypeptide). Such processes can improve the sequencing a single isolated polypeptide (e.g., by allowing fragments from different regions of the isolated polypeptide and/or different instances of the isolated polypeptide to be marked with respective different regionally-specific polynucleotide barcodes) and/or improve the sequencing of a sample that includes a mixture of different polypeptides (e.g., by allowing fragments from different polypeptides to be marked with respective different regionally-specific polynucleotide barcodes)

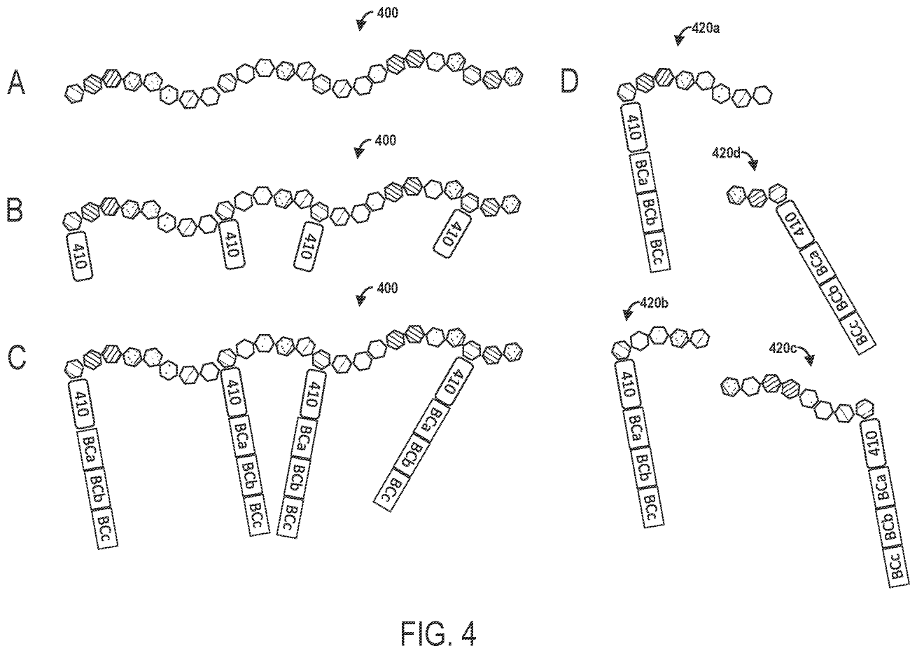

FIG. 4 illustrates aspect of an example process for creating regionally-specific barcodes on fragments of a target polypeptide such that fragments from the same target polypeptide or contiguous region thereof have coupled thereto the same regionally-specific barcode while other polypeptides or polypeptide fragments exhibit different regionally-specific barcode(s). Step “A” illustrates the target polypeptide 400. The target polypeptide 400 is a strand of amino acids (depicted as hexagons in FIG. 4) covalently coupled together via peptide bonds. The identity of the different amino acids is illustrated by different fill patterns. The target polypeptide could be the entirety of a protein or other polypeptide or other naturally-terminated polypeptide or could be a specified portion thereof, e.g., a specified subunit or other specified locus within a larger polypeptide. Additionally or alternatively, the target polypeptide could be a randomly-terminated fragment of such a naturally-terminated polypeptide or portion thereof. For example, the target polypeptide 400 could be a fragment of a polypeptide extending from one instance of a particular amino acid within the polypeptide to immediately before the next instance of the particular amino acid within the polypeptide (generated, e.g., by specifically digesting the polypeptide at each instance of the particular amino acid within the polypeptide).

The target polypeptide 400 could be isolated and/or purified such that it is the only polypeptide present in a sample. Alternatively, the target polypeptide 400 could be one of a plurality of different polypeptides (e.g., other proteins or fragments thereof, other isoforms of a protein and/or alternative translations of the same RNA) present in a sample.

Step “B’ of FIG. 4 illustrates the target polypeptide 400 after a plurality of probes 410 have been coupled to respective different amino acids of the target polypeptide 400. The probes 410 could include dsDNA, ssDNA, RNA, or some other polynucleotide (containing natural and/or modified nucleotides) that can be attached to amino acid side chains at a first end (e.g., a 3′ end of an ssDNA) and that can have additional barcode sequences ligated thereto at a second end (e.g., at a phosphorylated 5′ end). Attachment of the probes 410 to the amino acids could be specific to particular amino acids of the target polypeptide 400 (as depicted in FIG. 4) or could be nonspecific to more than one type of amino acid, or even to any amino acid, of the target polypeptide 400. Attachment of the probes 410 to the amino acids could include using ‘click’ chemistry or some other means to specifically or non-specifically attach the probes (e.g., a 3′ end of an ssDNA or RNA probe or a 3′ end of one strand of a dsDNA probe) to the amino acids of the target polypeptide 400 (e.g., to specifically targetable aspects of the side chain of one or more specified amino acids). The probes 410 could be attached to the amino acids directly or via a linking agent (e.g, a length of PEG or of some other polymer substance).

Regionally-specific polynucleotide barcodes could then be sequentially added onto the probes 410 using the methods described elsewhere herein. For example, repeated cycles of split-pool ligation of barcode sequences could be used in order to quickly and efficiently ‘grow’ respective different regionally-specific barcodes onto a plurality of different polypeptides and/or fragments of polypeptides. Step “C” of FIG. 4 shows the result of three cycles of ligation onto the probes 410 such that each probe 410 attached to the target polypeptide 400 has been extended to include first (“BCa”), second (“BCb”), and third (“BCc”) barcodes. These three barcodes in order represent the “regionally-specific barcode” that likely uniquely identifies the target polypeptide 400.

In some examples, the target polypeptide 400 could be digested (e.g., at the location of a subset of the amino acids to which the probes 410 are attached) subsequent to extending the probes 410 by one or more barcode sequences. Digestion methods can include one or more of applying trypsin, applying LysC, applying an enzymatic digestion process, or some other digestion process. After the digestion, the probes 410 could be further extended, thereby ‘growing’ sub-regional barcodes on probes attached to the different fragments of the target polypeptide 400 following the digestion.

Addition of barcode sequences onto the probe 410 can include a variety of substances and processes according to the composition of the probe 410. For example, if the probe 410 is composed of dsDNA, T7 ligase, T4 ligase, or some other ligase could be used to ligate additional barcode sequences onto the probes 410. In some examples, the probes 410 and each of the added barcode sequences could terminate in cycle-specific recognition sequences to facilitate the ligation process and to assist in preventing and/or facilitating the detection of instances where a particular probe failed to be extended as expected from exposure to one cycles of barcode addition. In examples where the probes 410 are composed of ssDNA or RNA, additional ssDNA or RNA barcodes can be ligated onto the probes 410 using an ssDNA or RNA ligase (e.g., RNA ligase RtcB). Incubation times, barcode sequences, and concentrations can be specified to reduce the likelihood that more than one instance of a barcode is ligated onto the probes 410 in any particular ligation cycle. However, the presence of such repeated ligation can later be detected in read sequences of the extended probe and accounted for, particularly in circumstances where the barcode sequences are not reused between ligation cycles. To facilitate such detection, barcodes can be designed to be rotationally invariant. In another example, ‘click’ chemistry could be used to sequentially attach barcode sequences to extend the probes 410. To prevent nonspecific binding between cycles of barcode attachment, orthogonal click chemistry reactions could be used in adjacent cycles. In yet another example, techniques employing ultraviolet light exposure to connect barcode sequences could be used. Multiple methods could be employed in sequence or in combination.

Once the probes 410 have been extended by a desired number of barcode sequences (thereby creating a regionally-specific barcode sequence), the target polypeptide 400 can be digested into fragments such that each fragment includes at least one of the extended probes 410. As shown in Step “D” of FIG. 4, this can include digesting the target polypeptide 400 at each amino acid to which a probe 410 is attached, such that each polypeptide fragment 420a, 420b, 420c, 420d of the target polypeptide 400 has a respective extended probe 410 attached to terminal amino acid of the fragment (e.g., to a C-terminal amino acid or to an N-terminal amino acid). Each of the fragments 420a, 420b, 420c, 420d could then be sequenced in tandem with its associated extended probe 410 and the regionally-specific barcode sequences of the extended probes used to associate the polypeptide fragment sequences with each other (e.g., with the same source polypeptide, from the same region within the same course polypeptide). This association can be used to speed and/or reduce the computational cost of alignment of the polypeptide fragment sequences by allowing the polypeptide fragment sequences to be ‘pre-aligned’ using their association according to the regionally-specific barcode sequences. Additionally or alternatively, this association can also be used to generate higher-accuracy polypeptide reconstructions by leveraging the distal sequence information represented by the regional association between the polypeptide fragment sequences.

Tandem sequencing of the polypeptide fragments and associated polynucleotide barcodes could be accomplished in a variety of ways. In some examples, the polypeptide fragments and associated polynucleotide barcodes could be affixed to a common support (e.g., a microbead, a glass slide), though the methods described herein can also be accomplished in solution without the use of a solid substrate. Affixing the polypeptide fragments and associated polynucleotide barcodes to the support could include attaching oligonucleotide foundation sequences to both of the polypeptide fragments the polynucleotide barcodes and then attaching those foundation oligonucleotides to adapter oligonucleotides already affixed to the solid support.

Decoupling the polypeptide fragments from their associated polynucleotide barcodes (e.g., to facilitate sequencing of the barcodes, and potentially additional polynucleotide sequences ligated on to encode the fragment polypeptide sequence, following sequencing of the polypeptide) could be done, e.g., by incorporating a restriction site within the probes 410 and, subsequent to affixing the polypeptides and associated polynucleotide barcodes to the support, restriction enzymes could be used to fragment the probes 410 at the restriction site. In another example, the polypeptides could have been coupled to their associated polynucleotide barcodes using a click chemistry reaction, and so another click chemistry reaction could be employed to decouple them. Detaching the polypeptide fragment from the associated regionally-specific polynucleotide could be done prior to sequencing one or both of them (e.g., using Edman degradation to sequence the polypeptide, and using NGS techniques or some other method to sequence the polynucleotide barcode)).

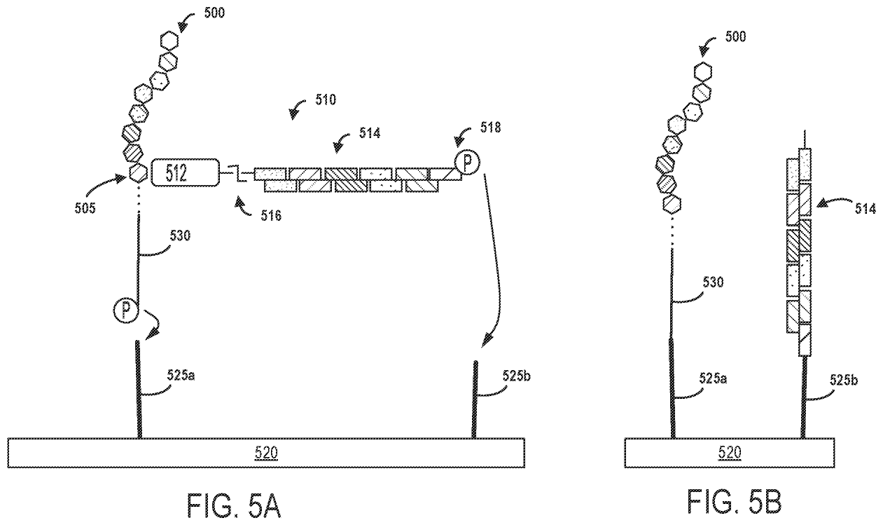

FIGS. 5A and 5B illustrate, by way of example, methods for affixing polypeptide fragments and their associated polynucleotide barcodes to the same support and then decoupling them from each other. As shown in FIG. 5A, adapter oligonucleotides 525a, 525b are already affixed to a solid support 520. A polypeptide fragment 500 is attached, via a terminal amino acid 505 (e.g, a C-terminal amino acid), to a handle 512 portion of a probe 510. The probe also includes a regionally-specific polynucleotide barcode 514 that is attached to the handle 512 via a restriction sequence 516. The probe 510 has been extended to include a 5′ phosphorylated oligonucleotide linker sequence 518. A 5′ phosphorylated foundation oligonucleotide 530 is attached to the terminal amino acid 505. The foundation oligonucleotide 530 and linker sequence 518 are then coupled to respective adapter oligonucleotides 525a, 525b (which may be the same or different), thereby affixing the polypeptide fragment 500 and the regionally-specific polynucleotide barcode 514 to the support 520. Coupling the foundation oligonucleotide 530 to the terminal amino acid 505 could include adding a sample of the polypeptide (e.g., a sample suspended in a 3M sodium acetate buffer solution such that the protein has a concentration of 3 mM) to a solution of 4-ethynylbenzaldehyde (e.g., a 10 mM solution of 4-ethynylbenzaldehyde suspended in methanol) and a solution of sodium cyanoborohydride (e.g., a 100 mM solution of sodium cyanoborohydride suspended in a 3M sodium acetate buffer solution) and shaking and heating the combined solution. For example, 33 uL of the 3 mM polypeptide solution could be combined with 1 uL of 10 mM 4-ethynylbenzalehyde solution and 66 uL of 100 nM sodium cyanoborohydride solution and placed on a shaking heat block set to 37 degrees Celsius and 1200 rpm for several hours (e.g., overnight). The polypeptide fragment 500 and the regionally-specific polynucleotide barcode 514 can then be decoupled from each other by fragmenting the probe 510 at the restriction site 516 (shown in FIG. 5B).

In some examples, sequencing the polypeptide fragment (e.g., 500) could include extending the associated polynucleotide barcode (e.g., 514) in a manner that is indicative of the sequence of the polypeptide fragment, and then sequencing the polynucleotide barcode. This has the benefit of reducing human or automatic sample handling effort/steps, allowing for increased density of polypeptide sequences on a plate or some other solid support, and easier and more accurate correspondence between the polypeptide sequences and the associated region-specific polynucleotide barcodes (since the polypeptide sequence will be represented by a portion of the same polynucleotide that includes the region-specific polynucleotide barcode).

This could be accomplished by using adding an aptamer or other substance that results in the ligation, onto the polynucleotide barcode, of an additional polynucleotide sequence that is indicative of the identity of one or more amino acids at the exposed terminus (e.g., N-terminus) of the polypeptide fragment that is opposite to the terminus of the polypeptide fragment via which the polypeptide fragment is attached to the support. In some examples, the an amino-acid-specific aptamer could terminate in a corresponding amino-acid-specific polynucleotide sequence. This amino-acid-specific polynucleotide sequence could then be ligated onto an exposed end of the polynucleotide barcode, after which the amino-acid-specific polynucleotide sequence can be fragmented away from the remainder of the aptamer (e.g., via fragmentation at a restriction site). The amino-acid-specific substance can then be washed, the terminal amino acid(s) removed (e.g., via Edman degradation, protease/enzymatic digestion, or via some other degradation method), and the process repeated until the entire sequence of the polypeptide fragment has been ‘transcribed’ into a representative polynucleotide sequence extended onto the region-specific polynucleotide barcode. The polynucleotide can then be sequenced to read both the region-specific barcode sequence as well as the sequence indicative of the polypeptide sequence. The contents of International Application PCT/US20/50574, filed Sep. 11, 2020, whose contents are incorporated by reference herein, includes a variety of such methods for ‘transcribing’ the sequence of a polypeptide onto a neighboring polynucleotide.

### VII. Example Methods

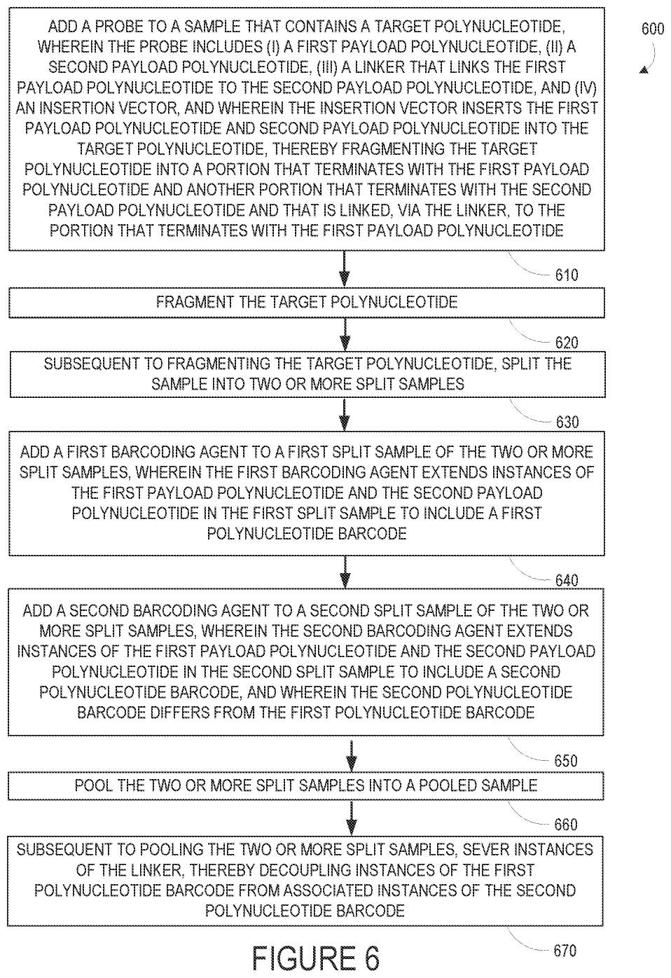

FIG. 6 depicts an example method 600. The method 600 includes adding a probe to a sample that contains a target polynucleotide, wherein the probe includes (i) a first payload polynucleotide, (ii) a second payload polynucleotide, (iii) a linker that links the first payload polynucleotide to the second payload polynucleotide. and (iv) an insertion vector, and wherein the insertion vector inserts the first payload polynucleotide and second payload polynucleotide into the target polynucleotide, thereby fragmenting the target polynucleotide into a portion that terminates with the first payload polynucleotide and another portion that terminates with the second payload polynucleotide and that is linked, via the linker, to the portion that terminates with the first payload polynucleotide (610). The method 600 additionally includes fragmenting the target polynucleotide (620). The method 600 additionally includes, subsequent to fragmenting the target polynucleotide, splitting the sample into two or more split samples (630). The method 600 additionally includes adding a first barcoding agent to a first split sample of the two or more split samples, wherein the first barcoding agent extends instances of the first payload polynucleotide and the second payload polynucleotide in the first split sample to include a first polynucleotide barcode (640). The method 600 additionally includes adding a second barcoding agent to a second split sample of the two or more split samples, wherein the second barcoding agent extends instances of the first payload polynucleotide and the second payload polynucleotide in the second split sample to include a second polynucleotide barcode, and wherein the second polynucleotide barcode differs from the first polynucleotide barcode (650). The method 600 additionally includes pooling the two or more split samples into a pooled sample (660). The method 600 additionally includes, subsequent to pooling the two or more split samples, severing instances of the linker, thereby decoupling instances of the first polynucleotide barcode from associated instances of the second polynucleotide barcode (670). The method 600 could include additional steps or features.

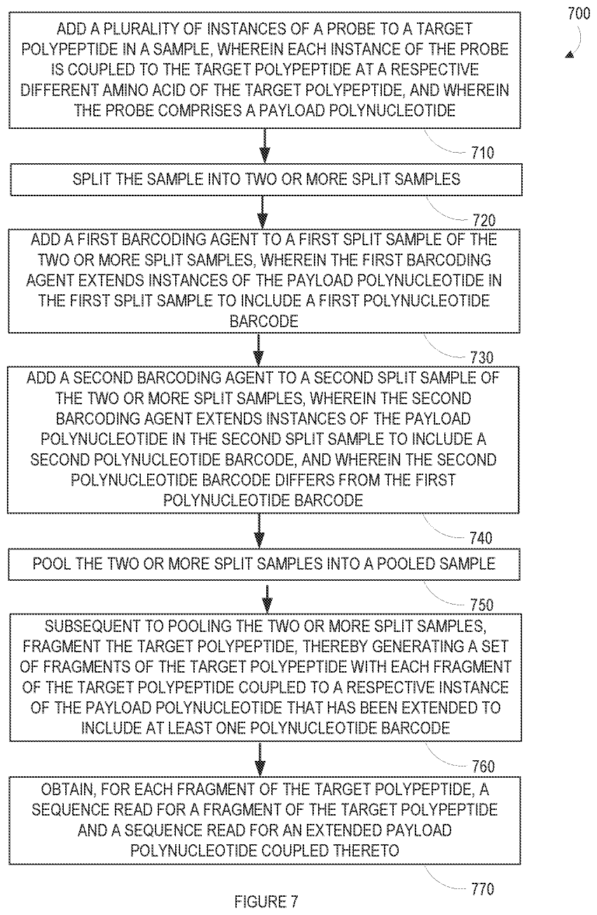

FIG. 7 depicts an example method 700. The method 700 includes adding a plurality of instances of a probe to a target polypeptide in a sample, wherein each instance of the probe is coupled to the target polypeptide at a respective different amino acid of the target polypeptide, and wherein the probe comprises a payload polynucleotide (710). The method 700 additionally includes splitting the sample into two or more split samples (720). The method 700 additionally includes adding a first barcoding agent to a first split sample of the two or more split samples, wherein the first barcoding agent extends instances of the payload polynucleotide in the first split sample to include a first polynucleotide barcode (730). The method 700 additionally includes adding a second barcoding agent to a second split sample of the two or more split samples, wherein the second barcoding agent extends instances of the payload polynucleotide in the second split sample to include a second polynucleotide barcode, and wherein the second polynucleotide barcode differs from the first polynucleotide barcode (740). The method 700 additionally includes pooling the two or more split samples into a pooled sample (750). The method 700 additionally includes, subsequent to pooling the two or more split samples, fragmenting the target polypeptide, thereby generating a set of fragments of the target polypeptide with each fragment of the target polypeptide coupled to a respective instance of the payload polynucleotide that has been extended to include at least one polynucleotide barcode (760). The method 700 additionally includes obtaining, for each fragment of the target polypeptide, a sequence read for a fragment of the target polypeptide and a sequence read for an extended payload polynucleotide coupled thereto (770). The method 700 could include additional steps or features.

It should be understood that arrangements described herein are for purposes of example only. As such, those skilled in the art will appreciate that other arrangements and other elements (e.g. machines, interfaces, operations, orders, and groupings of operations, etc.) can be used instead of or in addition to the illustrated elements or arrangements.

### VIII. Conclusion

It should be understood that arrangements described herein are for purposes of example only. As such, those skilled in the art will appreciate that other arrangements and other elements (e.g. machines, interfaces, operations, orders, and groupings of operations, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and implementations have been disclosed herein, other aspects and implementations will be apparent to those skilled in the art. The various aspects and implementations disclosed herein are for purposes of illustration and are not intended to be limiting, with the true scope being indicated by the following claims, along with the full scope of equivalents to which such claims are entitled. It is also to be understood that the terminology used herein is for the purpose of describing particular implementations only, and is not intended to be limiting.

1. A method, comprising:
adding a probe to a sample that contains a target polynucleotide, wherein the probe comprises (i) a first payload polynucleotide, (ii) a second payload polynucleotide, (iii) a linker that links the first payload polynucleotide to the second payload polynucleotide, and (iv) an insertion vector, and wherein the insertion vector inserts the first payload polynucleotide and second payload polynucleotide into the target polynucleotide, thereby fragmenting the target polynucleotide into a portion that terminates with the first payload polynucleotide and another portion that terminates with the second payload polynucleotide and that is linked, via the linker, to the portion that terminates with the first payload polynucleotide;
fragmenting the target polynucleotide;
subsequent to fragmenting the target polynucleotide, splitting the sample into two or more split samples;
adding a first barcoding agent to a first split sample of the two or more split samples, wherein the first barcoding agent extends instances of the first payload polynucleotide and the second payload polynucleotide in the first split sample to include a first polynucleotide barcode;
adding a second barcoding agent to a second split sample of the two or more split samples, wherein the second barcoding agent extends instances of the first payload polynucleotide and the second payload polynucleotide in the second split sample to include a second polynucleotide barcode, and wherein the second polynucleotide barcode differs from the first polynucleotide barcode;
pooling the two or more split samples into a pooled sample; and
subsequent to pooling the two or more split samples, severing instances of the linker, thereby decoupling instances of the first polynucleotide barcode from associated instances of the second polynucleotide barcode.

2. The method of claim 1, further comprising:
splitting the pooled sample into two or more additional split samples;
adding a third barcoding agent to a third split sample of the two or more additional split samples, wherein the third barcoding agent extends instances of the first payload polynucleotide and the second payload polynucleotide in the third split sample to include a third polynucleotide barcode; and
adding a fourth barcoding agent to a fourth split sample of the two or more additional split samples, wherein the fourth barcoding agent extends instances of the first payload polynucleotide and the second payload polynucleotide in the fourth split sample to include a fourth polynucleotide barcode, and wherein the fourth polynucleotide barcode differs from the third polynucleotide barcode.

3. The method of claim 2, wherein the first payload polynucleotide and the second payload polynucleotide of the probe each end in a first recognition sequence, wherein the first barcoding agent specifically targets the first recognition sequence to extend instances of the first payload polynucleotide and the second payload polynucleotide in the first split sample to include the first polynucleotide barcode and to end in a second recognition sequence, wherein the second barcoding agent specifically targets the first recognition sequence to extend instances of the first payload polynucleotide and the second payload polynucleotide in the second split sample to include the second polynucleotide barcode and to end in the second recognition sequence, wherein the third barcoding agent specifically targets the second recognition sequence to extend instances of the first payload polynucleotide and the second payload polynucleotide in the third split sample to include the third polynucleotide barcode, and wherein the fourth barcoding agent specifically targets the second recognition sequence to extend instances of the first payload polynucleotide and the second payload polynucleotide in the fourth split sample to include the fourth polynucleotide barcode.

4. The method of claim 2, further comprising:
prior to splitting the pooled sample into two or more additional split samples, fragmenting the target polynucleotide in the pooled sample.

5. The method of claim 1, wherein the first pay load polynucleotide and the second payload polynucleotide of the probe each end in a first recognition sequence, wherein the first barcoding agent specifically targets the first recognition sequence to extend instances of the first payload polynucleotide and the second payload polynucleotide in the first split sample to include the first polynucleotide barcode, and wherein the second barcoding agent specifically targets the first recognition sequence to extend instances of the first payload polynucleotide and the second pay load polynucleotide in the second split sample to include the second polynucleotide barcode.

6.-10. (canceled)

11. The method of claim 1, further comprising:
sequencing a plurality of segments of the target polynucleotide that include at least one of an instance of the first payload polynucleotide or an instance of the second payload polynucleotide to obtain reads of the fragments of the target polynucleotide; and
determining a sequence for the target polynucleotide based on the reads of the fragments of the target polynucleotide, wherein determining the sequence for the target polynucleotide comprises:
identifying a regional barcode for each of the read fragments of the target polynucleotide, wherein the regional barcode for a read fragment obtained from a fragment of the target polynucleotide that was present in the first split sample includes the first polynucleotide barcode, and wherein the regional barcode for a read fragment obtained from a fragment of the target polynucleotide that was present in the second split sample includes the second polynucleotide barcode; and
associating sets of the read fragments together based on correspondences between their respective identified regional barcodes.

12. (canceled)

13. The method of claim 1, wherein the target polynucleotide comprises RNA, wherein the target polynucleotide is a first isoform of an RNA sequence, and wherein the sample contains a second isoform of the RNA sequence, and wherein the first isoform differs from the second isoform.

14. (canceled)

15. A probe comprising:
a first payload polynucleotide;
a second payload polynucleotide;
a linker that links the first payload polynucleotide to the second payload polynucleotide; and
an insertion vector, wherein the insertion vector inserts the first payload polynucleotide and second payload polynucleotide into the target polynucleotide, thereby fragmenting the target polynucleotide into a portion that terminates with the first payload polynucleotide and another portion that terminates with the second payload polynucleotide and that is linked, via the linker, to the portion that terminates with the first payload polynucleotide.

16. (canceled)

17. The probe of claim 15, wherein the linker comprises polyethylene glycol with a length between 40 monomer subunits and 125 monomer subunits

18. The probe of claim 15, wherein the first payload polynucleotide includes a modified nucleotide via which the first payload polynucleotide is linked to the linker.

19. The probe of claim 15, wherein the probe additionally comprises a third payload polynucleotide that is associated with the first payload polynucleotide as double-stranded DNA and a fourth payload polynucleotide that is associated with the second payload polynucleotide as double-stranded DNA, wherein the insertion vector ligates the first payload polynucleotide to a 3′ end of a first strand of the target polynucleotide and ligates the third payload polynucleotide to a 5′ end of a second strand of the target polynucleotide, and wherein a portion of a 3′ end of the first payload polynucleotide that includes a first recognition sequence extends beyond a 5′ end of the third payload polynucleotide.

20. A method, comprising:
adding a plurality of instances of a probe to a target polypeptide in a sample, wherein each instance of the probe is coupled to the target polypeptide at a respective different amino acid of the target polypeptide, and wherein the probe comprises a payload polynucleotide;
splitting the sample into two or more split samples;
adding a first barcoding agent to a first split sample of the two or more split samples, wherein the first barcoding agent extends instances of the payload polynucleotide in the first split sample to include a first polynucleotide barcode;
adding a second barcoding agent to a second split sample of the two or more split samples, wherein the second barcoding agent extends instances of the payload polynucleotide in the second split sample to include a second polynucleotide barcode, and wherein the second polynucleotide barcode differs from the first polynucleotide barcode;
pooling the two or more split samples into a pooled sample;
subsequent to pooling the two or more split samples, fragmenting the target polypeptide, thereby generating a set of fragments of the target polypeptide with each fragment of the target polypeptide coupled to a respective instance of the payload polynucleotide that has been extended to include at least one polynucleotide barcode; and obtaining, for each fragment of the target polypeptide, a sequence read for a fragment of the target polypeptide and a sequence read for an extended payload polynucleotide coupled thereto.

21. The method of claim 20, further comprising:
splitting the pooled sample into two or more additional split samples;
adding a third barcoding agent to a third split sample of the two or more additional split samples, wherein the third barcoding agent extends instances of the payload polynucleotide in the third split sample to include a third polynucleotide barcode; and
adding a fourth barcoding agent to a fourth split sample of the two or more additional split samples, wherein the fourth barcoding agent extends instances of the payload polynucleotide in the fourth split sample to include a fourth polynucleotide barcode, and wherein the fourth polynucleotide barcode differs from the third polynucleotide barcode.

22. The method of claim 21, wherein the payload polynucleotide ends in a first recognition sequence, wherein the first barcoding agent specifically targets the first recognition sequence to extend instances of the payload polynucleotide in the first split sample to include the first polynucleotide barcode and to end in a second recognition sequence, wherein the second barcoding agent specifically targets the first recognition sequence to extend instances of the payload polynucleotide in the second split sample to include the second polynucleotide barcode and to end in the second recognition sequence, wherein the third barcoding agent specifically targets the second recognition sequence to extend instances of the payload polynucleotide in the third split sample to include the third polynucleotide barcode, and wherein the fourth barcoding agent specifically targets the second recognition sequence to extend instances of the payload polynucleotide in the fourth split sample to include the fourth polynucleotide barcode.

23. The method of claim 20, wherein the payload polynucleotide ends in a first recognition sequence, wherein the first barcoding agent specifically targets the first recognition sequence to extend instances of the payload polynucleotide in the first split sample to include the first polynucleotide barcode, and wherein the second barcoding agent specifically targets the first recognition sequence to extend instances of the payload polynucleotide in the second split sample to include the second polynucleotide barcode.

24.-28 (canceled)

29. The method of any of claim 20, further comprising:
subsequent to obtaining, for each fragment of the target polypeptide, a sequence read for the fragment of the target polypeptide and a sequence read for the extended payload polynucleotide coupled thereto, determining a sequence for the target polypeptide based on the sequence reads of the fragments of the target polypeptide, wherein determining the sequence for the target polypeptide comprises:
identifying a regional barcode for each of the sequence reads of the extended payload polynucleotides, wherein the regional barcode for a sequence read obtained from an extended payload polynucleotide that was present in the first split sample includes the first polynucleotide barcode, and wherein the regional barcode for a sequence read obtained from an extended payload polynucleotide that was present in the second split sample includes the second polynucleotide barcode; and
associating sets of sequence reads for the fragments of the target polypeptide together based on correspondences between regional barcodes identified in the extended payload polynucleotides associated therewith.

30. The method of claim 20, wherein fragmenting the target polypeptide comprises fragmenting the target polypeptide such that each instance of the payload polynucleotide that has been extended to include at least one polynucleotide barcode is coupled to a respective fragment of the target polypeptide via a first terminal amino acid of the fragment of the target polypeptide.

31. The method of claim 30, wherein obtaining a sequence read for a particular fragment of the target polypeptide comprises:
coupling the particular fragment to a support;
adding, to an extended payload polynucleotide that is associated with the particular fragment, a polynucleotide sequence indicative of an identity of at least one amino acid at an end of the particular fragment opposite the first terminal amino acid of the particular fragment; and
subsequent to adding the polynucleotide sequence indicative of the identity of the at least one amino acid at the end of the particular fragment opposite the first terminal amino acid, removing from the particular fragment at least one amino acid from the end of the particular fragment opposite the first terminal amino acid.

32. The method of claim 31, wherein adding the polynucleotide sequence indicative of an identity of at least one amino acid at an end of the particular fragment opposite the first terminal amino acid of the particular fragment comprises:
adding, to a sample that includes the support, an aptamer that selectively binds to polypeptides that terminate in the at least one amino acid that comprise the end of the particular fragment opposite the first terminal amino acid of the particular fragment, wherein the aptamer also comprises the sequence indicative of the identity of the at least one amino acid at the end of the particular fragment opposite the first terminal amino acid of the particular fragment; and
fragmenting, from the remainder of the aptamer, the sequence indicative of the identity of the at least one amino acid at the end of the particular fragment opposite the first terminal amino acid of the particular fragment.

33. The method of claim 31, wherein the payload polynucleotide comprises a restriction sequence, and wherein the method further comprises:
coupling an extended payload polynucleotide that is coupled to the particular fragment to the support; and
fragmenting the extended payload polynucleotide that is coupled to the particular fragment at the restriction sequence, thereby decoupling a portion of the extended payload polynucleotide that has been extended to include at least one polynucleotide barcode from the particular fragment.

34.-35. (canceled)
